(19) United States
(12) Patent Application Publication
(10) Pub. No.: US 2004/0267320 A1
(43) Pub. Date: Dec. 30, 2004

(54) Direct cortical control of 3d neuroprosthetic devices

(76) Inventors: Dawn M. Taylor, Shaker Heights, OH (US); Andrew B. Schwartz, Pittsburgh, PA (US)

Correspondence Address: Thomas D MacBlain, Gallagher & Kennedy, 2575 East Camelback Road, Phoenix, AZ, 85016-9225, US

(21) Appl. No.: 10/495,207

(22) PCT Filed: Nov. 12, 2002
(86) PCT No.: PCT/US02/36652

Related U.S. Application Data
(60) Provisional application No. 60/350,241, filed on Nov. 10, 2001.
(60) Provisional application No. 60/355,558, filed on Feb. 6, 2002.

(51) Int. Cl. A61N 1/00

(52) U.S. Cl. 607/2

(57) ABSTRACT

Control signals for an object are developed from the neuron-originating electrical impulses detected by arrays of electrodes chronically implanted in a subject's cerebral cortex at the pre-motor and motor locations known to have association with arm movements. Taking as an input the firing rate of the sensed neurons or neuron groupings that affect a particular electrode, a coadaptive algorithm is used. In a closed-loop environment, where the animal subject can view its results, weighting factors in the algorithm are modified over a series of tests to emphasize cortical electrical impulses that result in movement of the object as desired. At the same time, the animal subject learns and modifies its cortical electrical activity to achieve movement of the object as desired. In one specific embodiment, the object moved was a cursor portrayed as a sphere in a virtual reality display. Target objects were presented to the subject, who then proceeded to move the cursor to the target and receive a reward. In a noncoadaptive use of the algorithm as previously modified by a co-adaptation, unlearned targets were presented in the virtual reality system and the subject moved the cursor to these targets. In another embodiment, a robot arm was controlled by an animal subject.

### CROSS REFERENCE TO RELATED APPLICATIONS

Priority is claimed from U.S. provisional patent application Ser. No. 60/350,241 of Dawn M. Taylor and Andrew B. Schwartz, filed Nov. 10, 2001, entitled “Direct Cortical Control of 3D Neuroprosthetic Devices,” and Ser. No. 60/355,558 of Dawn M. Taylor and Andrew B. Schwartz, filed Feb. 6, 2002, entitled “Direct Cortical Control of 3D Neuroprosthetic Devices.” Both applications are incorporated herein by reference.

### STATEMENT OF GOVERNMENT FINANCIAL SUPPORT

Financial assistance for this project was provided by the U.S. Government, National Institute of Health Grant Numbers PHS N01-NS-6-2347 and PHS N01-NS-02321, and the United States Government may own certain rights to this invention.

### BACKGROUND

This invention relates to methods and apparatus for control of devices using physiologically-generated electrical impulses, and more particularly to such methods and apparatuses in which neuron electrical activity is sensed by electrodes implanted in or on an animal or a human subject and is translated into control signals adapted by computer program algorithm to control a prosthesis, a computer display, another device or a disabled limb.

Severely physically disabled individuals have, in the past, been afforded the opportunity to communicate or control devices using such physical abilities as they possessed. For example, individuals incapable of speaking, but capable of the use of a keyboard, have been afforded the opportunity to communicate by computer, computer keyboard and monitor. Those who have lost the use of their legs have been able to use hand control for either manually driven or motor operated wheelchairs. Tetraplegic individuals have been afforded the opportunity to control, for example, a wheelchair using mouth tubes into which they could blow. Such techniques are limited in their ability to afford the severely disabled the range of communications and activities of which such individuals are capable mentally. Moreover, certain mentally sound, but profoundly physically disabled individuals are what has been termed “locked in,” i.e. totally without ability to communicate or act.

It will be appreciated that it would be of immense value for the most severely physically disabled individuals to have methods and apparatus serving as an interface or transducer converting that individual's physiologically-generated electrical impulses to useable control signals. These then could be used for the control of e.g., a communication device, a prosthesis, a wheelchair, a computer program cursor or a video game portrayed on a computer monitor, or the muscles of a paralyzed limb.

Cortical neurons are known to modulate their activity prior to a subject's movement. Researchers have anticipated using these signals to control various devices directly [1, 2]. One of the difficulties with this approach is that many neurons can be needed to predict intended movement direction accurately enough to make this prediction practical. Estimates range from 40 to 600 cells or more [4, 7]. Prior studies made their estimates based on open-loop experiments by recreating arm trajectories from cortical data off-line. This prior work does not examine a closed-loop situation in which the subjects have visual feedback of the brain-controlled movement, allowing them to make on-line corrections by modifying their recorded activity.

As signal processing technology improved, many groups tested the feasibility of decoding the cortical signals in real time. Chapin, Moxon, Markowitz and Nicolelis (1999) [3] used both principle component analysis and recurrent artificial neural networks to decode in real time one-dimensional cortical signals during lever-push movements in a rat.

Wessberg, Stambaugh, Kralik, Beck, Laubach, Chapin, Kim, Biggs, Srinivasan, and Nicolelis (2000) [4] also used artificial neural networks as well as linear models to interpret motor, pre-motor and posterior parietal cortex activity in real time. They did ‘real time’ trajectory creation offline using data from multiple days to simulate large recorded populations of cells. Their extrapolations from different cortical areas suggest between 376 and 1,195 cells would be needed to predict movements accurately (˜600 cells when taken from multiple areas together). They also used these real-time signals to control a robot. However, these experiments were open-loop with the animal receiving no feedback from the robot.

Close-loop real-time brain-control of movements in monkeys has been done by two research groups other than the inventors. Meeker et al. (2001) [25] controlled one-dimensional cursor movements using parietal cortex activity. Serruya, Hatsopoulos, Paninski, Fellows and Donoghue (2002) [30] used linear filters to produce two-dimensional brain-controlled cursor movements in one monkey. Their monkey was able to successfully get a brain-controlled cursor to random targets at a speed close to that achieved with the cursor under hand-control. A high cursor gain allowed the brain-controlled cursor to move fast. However, there was little precision or endpoint control in the movements. Their best published trajectories overshot and oscillated around the targets before finally hitting them. Holding the cursor stationary in the target was not a requirement of the task.

Both the Meeker et al., and Serruya et al., along with our work, illustrate the value of visual feed back in neuroprosthetic control. Schnmidt, Bak, McIntosh, and Thomas (1977) [32] used operant conditioning to train monkeys to control the firing rates of individual motor cortex cells. Fetz and Finocchio (1975) [12] demonstrated that, with operant conditioning, motor cortex cells can be trained to alter their firing correlations to muscle activity. These closed-loop animal studies suggest a high level of trainability in cortical cells. This plasticity makes cortical cells very desirable as control signals.

Kennedy, Bakay, Moore, Adams, and Goldwaithe (2000) [20] have developed a neurotrophic electrode that triggers neurons to grow into an implanted glass cone electrode. This technique has enabled them to record from the same cells for an extended period of time. However, the number of recorded cells is low - about one or two per implant. Even with this small number of signals, this type of implant has allowed locked-in patients to communicate using the firing rates of these cells to scroll through and select letters from a list. This limited use of electrodes in the first human patients has shown that motor cortex cells can be trained to produce useful modulation patterns, even after long periods of inactivity.

### SUMMARY

In accordance with this invention, methods and apparatus are provided that can convert a subject's physiological electrical activity to movement of a real or virtual object in a manner discernible to the subject.

In a preferred embodiment, test subjects' cerebral cortex in the motor and pre-motor area were the locations from which electrical impulses were derived for development of electrical control signals applied to control devices. More broadly however, the techniques and apparatus of the invention should enable the development of electrical control signals based upon electrical impulses that are available from other regions of the brain, from other regions of the nervous system and from locations where electrical impulses are detected in association with actual or attempted muscle contraction and relaxation.

Advances in chronic recording electrodes and signal processing technology [3, 4] are used in accordance with the specific exemplary embodiment set out in detail below to employ cortical signals efficiently and in real time. The methods and apparatus of this invention provide electrical control signals to enable the use of cortical signals to, inter alia, move a computer cursor, steer a wheelchair, control a prosthetic limb or activate muscles in a paralyzed limb. This can provide new levels of mobility and productivity for the severely disabled.

In a specific preferred embodiment, the calculation of amount of the movement is a function of a firing rate of one or more neurons in a region of the brain of the subject. Although the inventors used a moving average of the firing rates of the cells, this invention could be used with other characteristics of the subject physiologically-generated electrical signals such as the amplitude of the local field potentials, the power in the different frequencies of the local field potentials, or the amplitude or frequency content of the muscle-associated electrical activity. In accordance with an algorithm used in the specific exemplary embodiment of the Detailed Description, to calculate the distance to move an object, a normalized firing rate in a time window is calculated. A digital processing device such as a computer or computerized controller applies the firing rate information to determine movement using the programmed algorithm. For at least a portion of the firing rates detected, a firing rate-related value is weighted by a “positive weighting factor” if the measured rate is greater than a mean firing rate and is weighted by a negative factor if the rate is less the mean firing rate. The moveable object then is moved a distance depending on at least a portion of the weighted firing rate-related value.

“Positive and negative weighting factors” as used herein mean weighting factors that are applied to weight of a particular unit's electrical input to the algorithm. That sums those individual inputs to either enhance or diminish the contribution by the particular unit in the calculation of the object's movement. The “positive” weighting factor is a weighting factor, either positive or negative in value, that is used when the normalized value electrical signal-derived value of an algorithm input for a particular unit is above zero, hence “positive.” The normalized value is the measured value minus a mean value of the algorithm input. The “negative” weighting factor is a weighting factor, either positive or negative in value, that is used when the normalized value of the electrical signal-derived value of the algorithm input for a particular unit is below zero, hence “negative.” Specific examples are given in connection with the exemplary embodiment of the Detailed Description where the electrical signal-derived value is the unit's firing rate.

In the exemplary embodiment of the following Detailed Description, an array of electrodes is implanted in a subject's cerebral cortex in the motor and pre-motor areas of the brain. Neuron-generated electrical signals are transmitted to the computerized processing device. That device, may be a computer, a computerized prosthetic device or an especially adapted interface capable of digital processing. It may be used to activate nerves that contact the muscles of a disabled limb. Typically, in accordance with the preferred embodiment, the object to be controlled by the subject is moved in the visual field of the subject. For example, where the object is a movable computer display object such as a cursor, this “virtual” object is portrayed in a computer display environment in the visual field of the subject. In the case of an animal subject, such as the monkeys used in the tests described below, those subjects are allowed to move the cursor first by hand, using a motion detector attached to the monkey's arm. This familiarizes the subject with the task at hand. Then the subject's arms are restrained. In each case, for the purpose of reinforcement, the subject may be afforded a reward upon achievement of a predetermined, desired movement of the object.

For use in detecting both the described and other physiological electrical impulses in other embodiments of this invention using other regions of the brain or body, the firing of the neurons may be detected either from the same electrode arrays, electrodes placed on the surface of the cortex on the surface of the scalp, or imbedded into the skull, or electrodes in the vicinity of peripheral nerves and/or muscles. Electrical characteristics other than firing rate that can prove useful in this context are: a) normalized local field potential voltages; b) normalized power in the various frequency bands of the local field potentials; and c) normalized muscle electrical activity (rectified and/or smoothed voltage amplitude or power in different frequency bands) in all cases. Local field potentials are slower fluctuations in voltage due to the changes in ion concentrations related to post synaptic potentials in the dendrites of many neurons as opposed to the firing rate which is a count of the action potentials in one or a few recorded cells in a given time window. This invention's algorithm could also be used with the recorded electrical activity of various muscles. Some muscles show electrical activity with attempted contraction even if it's not enough to produce physical movement. Any or all of these types of signals can be used in combination. Researchers have shown that local field potentials and muscle activity can be willfully controlled. Here the invention provides a markedly improved way of translating these signals into multi-dimensional movements. The type of signals to go into the coadaptive algorithm can be quite broad, although firing rates are used as the electrical characteristic of the sensed electrical impulses in the exemplary embodiment of the Detailed Description.

In the use of any of the selected characteristics of the detected electrical impulses, a similar normalization is employed. That is, subtracting the means (calculated either as a stationary value from previously recorded data or taken over a large moving window) and dividing by some value which will standardize the range of values (e.g. by one or two standard deviations, again that can be calculated either as a stationary value from previously recorded data or taken over a large moving window).

From detected electrical impulses, inputs to a computational processor are developed. By “computational processor” is meant, without limitation, a PC, a general purpose computer, a digital controller with digital computational capability, a micro-processor controlled “smart” device or another digital or analog electrical unit capable of running programmed algorithms like those described here. The processor applies the characteristics of the detected electrical impulses to develop a signal with representations of distance and direction. In the visual field of the subject, the object moves a distance and in a direction represented by the calculated signal. From the firing rates or other variable characteristics of the electrical impulses from the neurons or neuron groups, the algorithm provided in the programming of the computational processor develops the signals used to control the “object.” “Object, as used herein, means a real or virtual thing or image, a device or mechanism without limitation.

In the coadaptive technique, subjects train and learn to move the object while the algorithm is adapted to improve the subject's results. Weighting factors are employed to emphasize movement of the object in a “correct” direction. Each electrical signal (e.g. firing rate, local field potential voltage or frequency power, etc.) is assigned a set of positive and negative weights which are used when the signal is above or below its mean respectively. (Either of these weights may be positive or negative values.) The magnitude of these weights are adjusted to allow cells which are producing more useful movement information to contribute more to the movement. Having different positive and negative weights also allows for cells to contribute differently in different parts of their firing range. In the specific preferred embodiment described below, weights are iteratively adjusted in a way that minimizes the error between the actual movement produced and the movement needed to make the desired movement. The coadaptive technique has been employed to develop control signals that worked well for a particular subject.

In tests of the invention, rhesus macaques learned to control a cursor in a virtual reality display as the programmed algorithm adapted itself to better use the animal's cortical cell firings. In the coadaptive procedure, the firing rates of the macaques' neurons in the cortex in pre-motor and motor regions of the brain known to affect arm movement were employed. Moving averages of the firing rates of cells, continually being updated, were used as inputs to a coadaptive algorithm that converted the detected firing rates to instructions (or control signals) that moved a cursor in a virtual reality display. Targets were presented to the animals who successfully learned to move the cursor to the presented targets consistently. The coadaptive algorithm was continually revised to better achieve “goal” movement, i.e. the desired movement of cursor to target.

In a noncoadaptive procedure, the algorithm refined by the coadaptive technique is employed to enable the subject to control the object. In one example, the subject, again, a rhesus macaque, was able to move a cursor to targets for which he had not trained during the coadaptive procedures. In another application of the invention, a macaque successfully controlled a robot arm during both the coadaptive algorithm refinement and subsequently based on the refined algorithm. The macaque modified its approach to take into account the robot arm's differences in response (as compared to a cursor). The subject was able as well to effectively make long sequences for brain-controlled robot movements to random target position in 3D space.

The coadaptive algorithm worked well in determining an effective brain-control decoding scheme. However, it can be made more effective by incorporating correlation normalization terms. Also, adding an additional scaling function that more strongly emphasized units with similar positive and negative weight values will reduce the magnitude of the drift terms and result in more stability at rest.

The coadaptive algorithm can also be expanded into any number of dimensions. Additional dimensions can be added for graded control of hand grasp, or for independent control of all joints in a robotic or paralyzed limb. The coadaptive process can be expanded even further to directly control the stimulation levels in the various paralyzed muscles or the power to various motors of a robotic limb. By adapting stimulation parameters based on the resulting limb movement, the brain may be able to learn the complex nonlinear control functions needed to produce the desired movements.

The above and further objects and advantages of the invention will be better understood from the following detailed description of at least one preferred embodiment of the invention, taken in consideration with the accompanying drawings.

### BRIEF DESCRIPTION OF THE DRAWINGS

FIG. 1 is a diagrammatic illustration of a test subject in place before a virtual reality display operated in accordance with the present invention;

FIG. 1a is a diagrammatic illustration like that of FIG. 1 wherein the test subject has both arms restrained;

FIG. 2 is a diagrammatic representation of the elements of a virtual reality display portrayed to the test subject of FIG. 1;

FIG. 3 is a perspective view of an electrode array like those implanted in the cerebral cortex of the subject of FIG. 1;

FIG. 4a and 4b are diagrams indicating the location of electrode arrays in the brains of two test subjects in the preliminary background experiments;

FIG. 5 is an illustration of trajectories of subjects' cursor movement towards target presented in a virtual reality display like that illustrated in FIG. 1;

FIG. 6 is a graphical presentation of improvement in a pair of subjects' closed-loop minus open-loop target hit rate as a function of days of practice;

FIG. 7 is a diagram like those of FIGS. 4a and 4b indicating the location of electrode arrays in the brain of another subject used in tests of the present invention;

FIG. 8 is an illustration of cursor trajectories before and after coadaptation of the present invention;

FIG. 9 is a graphical representation of one subject's performance using the coadaptive method and apparatus of the invention;

FIG. 10 is a graphical representation of percentage of targets that would have been hit had the target size been larger in certain tests of the present invention;

FIG. 11 is a graphical illustration of a subject's performance after a 1-½ month hiatus;

FIG. 12 is a diagrammatic representation like that of FIG. 2 showing six additional virtual reality untrained target elements;

FIG. 13 is a series of representations of trajectories of cursor movement by subjects in a virtual reality setting like that of FIG. 5 using a noncoadaptive algorithm in a constant parameter prediction algorithm task;

FIG. 14 is a graphical illustration of two histograms (before and after regular practice) showing a number of cursor movements involved in successful sequences of movements;

FIG. 15 is a diagrammatic illustration like that of FIG. 1 and shows a test subject whose cortical neuron firing rate is used control a robot arm;

FIG. 16 is a pair of illustrations of trajectories of the robot arm of FIG. 18 under control of a subject's cortical neuron activity and shows trajectories from the coadaptive mode;

FIG. 17 is an illustration of trajectories of a subject's cursor movements to and from targets directly controlled by the subject's neuron firing and where a robot arm is used in a system like that of FIG. 18 on the left, and without a robot (direct cortical cursor control) on the right; and

FIG. 18 presents two graphical illustrations of success in a subject's hitting targets at a particular position and returning to the central start position of the cursor, as well as hitting just the target and also missing entirely.

### DETAILED DESCRIPTION

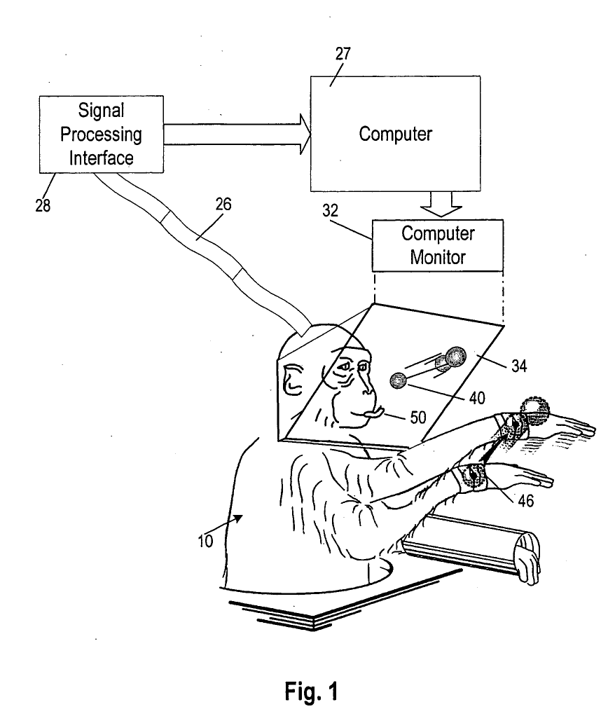

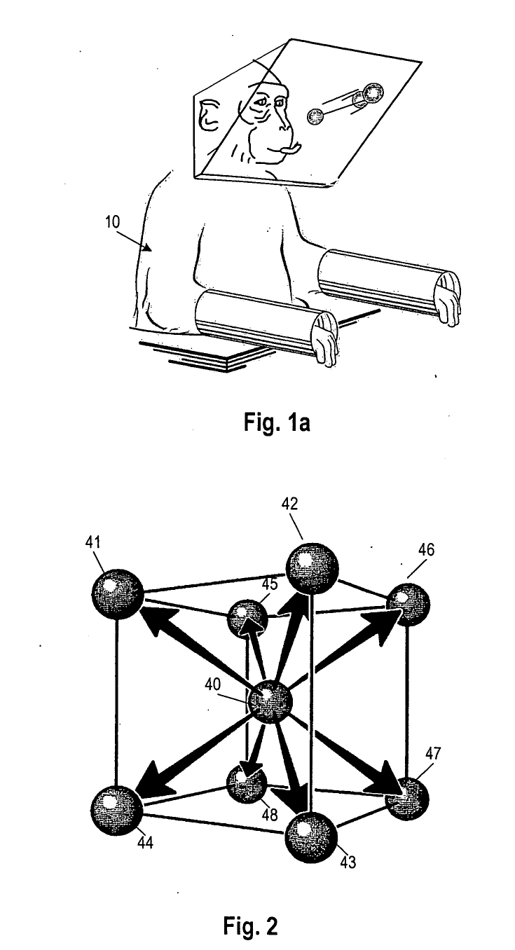

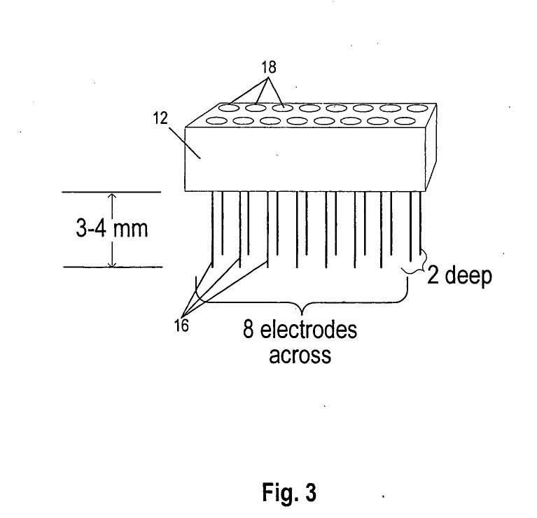

As illustrated generally in FIG. 1 and described in greater detail below, an animal subject 10, specifically a rhesus macaque, had implanted in an area of its brain known to control arm movement, four arrays of 16 closely spaced electrodes each. Such an array is depicted in FIG. 3. It includes an insulating support block 12, the thin conductive microwire electrodes 16 of three to four millimeters in length, and output connectors 18 electrically connected to the electrodes 16.

As illustrated in FIG. 1, conductors, shown as a ribbon 22 carried electrical impulses to a computer 26 via such interface circuitry 28 as was useful for presenting the impulses as useable to the computer inputs. In a virtual reality system, generally designated 29, the computer output 30 was used to drive computer monitor 32, which, after passage through a polarizing shutter screen was reflected as a three-dimensional display on a mirror 34. The subject 10 viewed the polarized mirror images through polarized left and right lenses to view a 3D image. A cursor 40 was projected onto the mirror 34. Its movement was under control of the computer 26.

As shown in FIG. 2, one of eight targets 41-48 was displayed for the subject to move the cursor 40 to under cortical control. Successful movement of the cursor 40 to whichever target was presented resulted in the subject animal 10 receiving a drink, as a reward, via a tube 50.

Background Experiments: Open Versus Closed-Loop Control

Until recently, most cortical control research consisted of recording neural activity during repeated trials of well-trained movements in behaving animals, then trying to reconstruct those movements from the cortical data afterwards. This technique is useful for comparing different decoding methods on the same data set. It is also necessary for testing decoding methods on larger numbers of units than were recorded simultaneously.

The earliest studies used acute movable electrodes to record one unit at a time. Georgopoulos et al. (1988) [5] showed that 3D arm trajectories could be recreated fairly accurately with a population vector using about 200 units. Salinas et al. (1994) [6] showed that an optimal linear estimation method could reduce the number of units needed to achieve the same accuracy. However, they never recreated full 3D trajectories using the optimal linear estimation method. Therefore, it's unclear just how much this method would reduce the number of units needed.

Lee et al. (1998) [7] recorded from seven movable electrodes simultaneously in primate motor cortex during a two dimensional center-out task. They then combined units from different recordings and predicted movement direction using the population vector and the optimal linear estimation methods. Their results suggest that only about 30 units were needed to accurately predict movement direction based on firing rates averaged over the whole movement. They also suggested that only about 40 units were needed to predict movement vectors throughout a trajectory using smoothed firing rates at about 50 msec intervals. Their work, however does not appear to take into consideration accumulation of errors over the length of the predicted movement.

Although these studies imply that it may be possible to accurately predict movement trajectories using 40 to 200 units, these results cannot be applied to chronic human implants. The units used in these studies do not accurately represent the quality of the units likely to be recorded from fixed multichannel electrode arrays in humans. These acute recordings used moveable electrodes which allowed the researcher to pick out well-isolated, well-tuned units. With fixed electrodes, the tuning quality, waveform amplitude, and the isolatability of the units are generally less favorable.

Wessberg et al. (2000) [4] did a more realistic extrapolation study using fixed multichannel electrodes in the motor, pre-motor and posterior parietal corticies of behaving monkeys. By combining data recorded from multiple days, they estimated the number of units needed to accurately predict 3D trajectories to range from 376 to 1,195, depending on the cortical area. They also estimated about 600 would be needed if all three areas were used.

This number of units exceeds the recording capacity of today's electrode technology (currently up to about 100 units), and would suggest that cortical control of neuroprosthetic devices is a technology for the distant future. However, these offline studies do not take into consideration that patients will usually have visual feedback of their brain-controlled movements. This visual feedback will allow the patient to correct errors in their trajectories as they happen. It also opens up the possibility that the patients will learn to modulate their cortical signals more effectively with practice. This could potentially lower the number of units needed for practical control of a prosthetic device, and make this technology a viable option with today's recording capabilities.

The effect of visual feedback on movements derived from cortical signals was examined by comparing ‘closed-loop’ trajectories, where the subjects had visual feedback of their brain-controlled trajectories, with ‘open-loop’ trajectories created offline from cortical activity recorded during normal center-out arm movements. This allowed comparison of the accuracy of 3D trajectories made from cortical signals with and without the benefit of visual feedback. The specific trajectories compared were decoded with the same movement prediction algorithm applied to the same recorded units from the same monkeys on the same day.

By repeating this experiment over many days, the inventors determined that visual feedback allowed the subjects to make long-term improvements in their brain-controlled movement skills with regular practice.

Methods

The virtual reality system of FIG. 1 was used to give each rhesus macaque 10 the experience of making brain-controlled and non-brain-controlled three-dimensional movements in the same environment. The animals made real and virtual arm movements in a computer-generated, 3D virtual environment by moving the cursor from a central-start position to one of eight targets located radially at the corners of an imaginary cube. The monkeys could not see their actual arm movements, but rather saw two spheres—one of the stationary ‘target’ (blue) sphere 41-48 and the mobile ‘cursor’ (yellow) sphere 40 with motion controlled either by the subject's hand position (“hand-control”) or by their real-time neural activity (“brain-control”).

The mirror 34 in front of the monkey's face reflected a 3D stereo image of the cursor and target projected from a computer monitor 32 above. On the other side of the mirror, the monkey moved one arm 52 with a position sensor 54 taped to the wrist. The 3D position of the cursor was determined by either the position sensor 54 (“hand-control”) or by the movement predicted by the subject's cortical activity (“brain-control”).

The movement task was a 3D center-out task. The cursor was held at the central position until a target appeared at one of the eight radial locations shown in FIG. 2 which formed the corners of an imaginary cube. The center of the cube was located distal to the monkey's right shoulder.

The image was controlled by an SGI Octane® workstation (available from Silicon Graphics, Inc., Mountain View, Calif., US) acting as the computer 26 in the image diagrammatic illustration of FIG. 1. The workstation is a UNIX workstation particularly suited to graphical representations. The subject 10 viewed the image through polarized lenses and a 96 Hz light-polarizing shutter screen which created a stereo view. 3D wrist position was sent to the workstation at 100 Hz from an Optotrak® 3020 motion tracking system 56. (Available from Northern Digital, Inc. Waterloo, Ontario, CAN) This system measures 3D motion and position by tracking markers (infrared light-emitting diodes) attached to a subject. Cortical activity was collected via a Plexon® Data Acquisition system, serving as the interface 28 of FIG. 1. (Available from Plexon, Inc., Dallas, Tex., US.) Spike times were transferred to the workstation 26, and a new brain-controlled cursor position was calculated every ˜30 msec.

Hand and brain-controlled movements were performed in alternating blocks of movements to all eight targets. The left arm was restrained while the right arm was free to move during both hand- and brain-controlled movement blocks. The cursor radius was 1 cm. Target and center radii were 1.2 cm. The liquid reward was given at the tube 50 when the cursor boundary crossed the target boundary for ˜300 ms or more. Radial distance (center start position to center of target) was 4.33 cm under brain-control. Since hand-controlled movements were quick, radial distance Was increased to 8.66 cm during the hand-controlled movement blocks to increase the duration of cortical data collection. For a fair comparison, of fine predicted trajectory (open-loop) hit rates were calculated with targets at the online brain-controlled distance of 4.33 cm. Each day's open-loop trajectories calculated offline were scaled, so the median radial endpoint distance was also 4.33 cm.

The subjects had 10 to 15 seconds to hit the targets. Therefore, on-line brain-controlled velocities were scaled down to ensure only reasonably direct trajectories could get to the target with in the 10-15 sec time limit.

Hand and brain-controlled movements were performed in alternating blocks of movements to all eight targets. However, under brain-control, missed targets were repeated up to five times per block for practice. Performance statistics were compiled by first averaging statistics from repeated targets together in each block to ensure statistics represented all targets equally, even if the number of movement samples differed between targets.

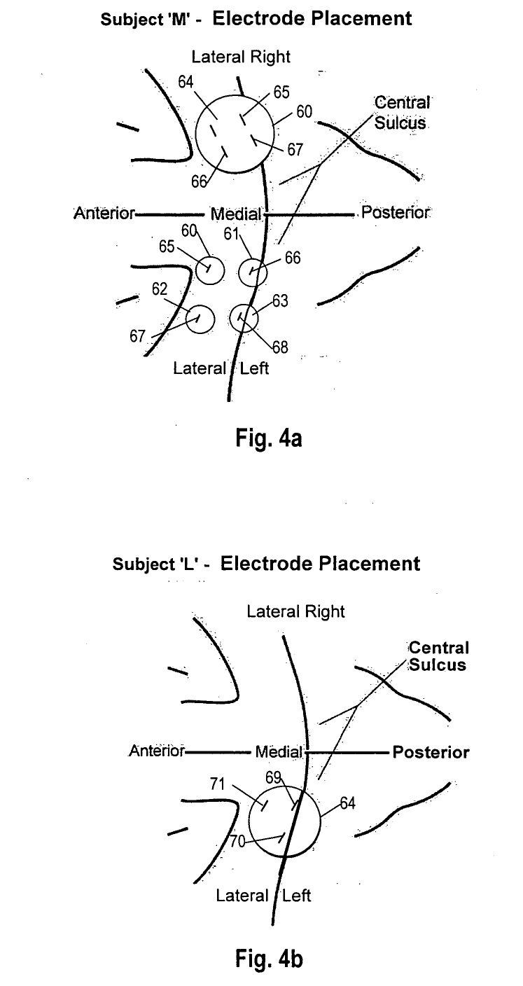

Monkeys ‘L’ and ‘M’ were chronically implanted in the motor and pre-motor areas of the left hemisphere with arrays 16 consisting of fixed stainless steel and/or tungsten microwires insulated with Teflon/polyimide. FIGS. 4a and 4b show estimated locations of the electrodes. The circles 60-63 and 64 represent craniotomies. Black straight lines 65-68 in subject ‘M,’ FIG. 4a, and 69-71 in subject ‘L,’ FIG. 4b, indicate approximate placement of arrays. Monitoring cortical activity during passive and active arm movements showed both animals had electrodes at units related to proximal and distal arm areas. Monkey ‘M’ also had some electrodes of arrays 71-74, at units related to upper back/neck activity (not relevant here). Many electrodes detected waveforms from multiple cells, some of which could not be individually isolated. The terms ‘cell’ or ‘unit,’ as used here, then, refer to both individually-isolated cells and inseparable multi-cell groups.

Often channels suspected of having very low amplitude cell activity (within the noise floor) were included in each day's experiment. Each unit's vectors were scaled based on tuning quality, so these noisy units usually had weights too small to significantly affect the population vector.

At the beginning of each day's experiments, 8-10 minutes of center-out arm movements and cortical data were collected to measure the baseline movement-related behavior of the recorded cells. This was then used to calculate the preferred directions and R² values used in the modified population vector.

Since new preferred directions and scaling factors were calculated each day, differing estimation errors (as well as any actual changes in the tuning properties) slightly changed the form of the population vector from day to day. Most of both animals' units had fairly stable waveforms and tuning properties between days. Larger variations between days in the modified population vector coefficients sometimes happened due to intermittent hardware problems making some recorded channels noisy or unusable.

Results and Discussion

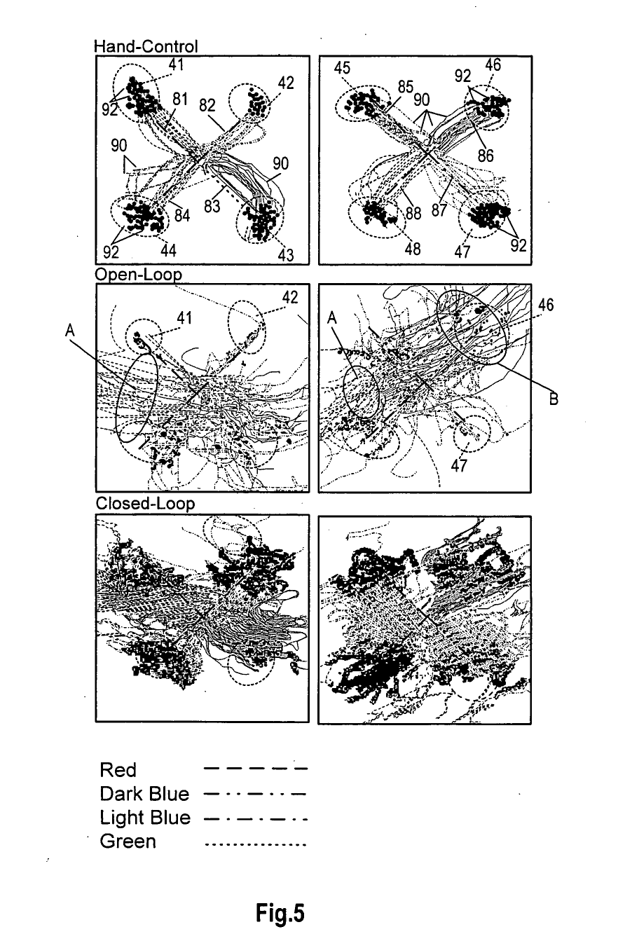

FIG. 5 shows examples of trajectories from this experiment. The top two figures show examples of actual hand trajectories to the eight targets. The eight thick straight lines 81-88 connect the cube center to the center of the eight targets 41-48 (generally indicated in FIG. 5 without being to scale). Thin lines 90 show the individual trajectories and are color coded by their intended target's line color discernable as varying shades of gray in FIG. 5's black and white reproduction. Black dots 92 indicate when the target was actually hit. The color coded figure more dramatically illustrates the results discussed here. A copy is being submitted for filing in the application file and is available on line at the website of Science magazine. The color scheme in each left hand plot is the same, the direct lines 81 and thin lines 90 directed toward the targets 41, 42, 43 and 44 are red, dark blue, green and light blue, respectively. The right hand plots are consistent with lines towards targets 45, 46, 47 and 48, light blue, green, dark blue and red, respectively.

The middle two plots of FIG. 5 show open-loop trajectories created offline from the cortical data recorded during the normal hand-controlled movements. There is some organization to these open-loop trajectories. Some target's trajectories are clustered together (e.g. red group dominating the area marked A in both plots and the green group dominating the area B in the right plot) while other groups show little organization, and covered little distance. This suggests the population vector did not accurately model the movement encoding of the cortical signals. On the day shown, only 22 units were recorded and only 17 were used after scaling down poorly-tuned units. With these results, it's not surprising that previous offline research suggested a few hundred units would be needed to accurately recreate aim trajectories.

The bottom row shows the closed-loop trajectories. Although they are not nearly as smooth as the normal hand trajectories, they did hit the targets more often than the open-loop trajectories. The subjects made use of visual feedback to redirect errant trajectories back toward the targets. In the closed-loop case, there were also more uniform movement amplitudes toward each of the targets. Although only small movements were made to the two dark blue targets 42, 47 in the open-loop case, the subject managed to make sufficiently-long trajectories in that direction to get to the targets under closed-loop brain-control. The trajectories, which extended beyond the targets in the open-loop case (e.g. left red, 41, and right green, 46, trajectories) were halted and redirected back to the targets in the closed-loop case.

This experiment was conducted with monkeys ‘L’ and ‘M’ for 32 and 40 days respectively. In both subjects, about 18 cells were used to create open- and closed-loop trajectories. As expected with so few cells, the open-loop trajectories were never very accurate. Although these trajectories went toward the correct targets more often than chance, they usually had at least one of the orthogonal X, Y or Z components pointing in the wrong direction.

Closed-loop trajectories often started out in the wrong direction, but were then redirected back to the correct octant. The closed loop trajectories hit the targets significantly more often than the open-loop trajectories in both animals.

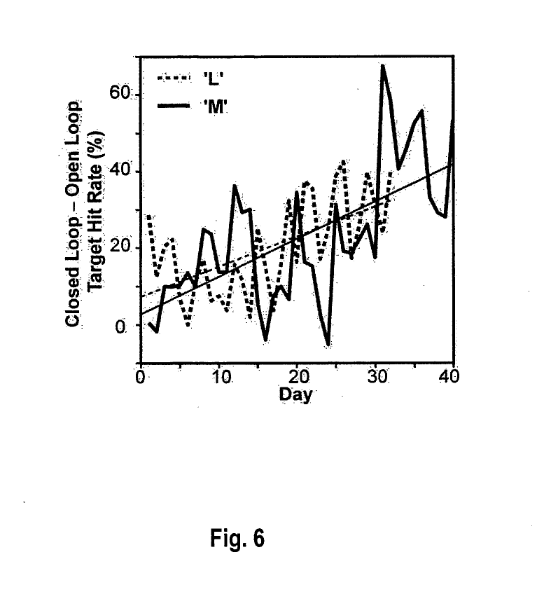

Both animals significantly improved their closed-loop target hit-rate over the course of the experiment. This might occur by chance if the number and/or quality of recorded cells improved during this time. If this were the case, one would also expect improvement in the off-line, open-loop success rate. Instead, the off-line hit-rate declined, suggesting that the subjects learned to modulate their brain signals more effectively with visual feedback in the closed-loop condition. FIG. 6 shows each animal's difference in target hit rate (closed-loop minus open-loop) as a function of the number of days of practice. The thin lines are the linear fits of the data. Subject ‘M’ showed an increase in closed-loop target hit rate of about 1% per day (P<0.0001) over the open-loop hit rate. Subject ‘L’ showed slightly less improvement—about 0.8% per day (P<0.003).

Most of the cortical units recorded were stable from day to day. Some were stable for more than two years (Williams, Rennaker, & Kipke, 1999). [40] Other units showed changes in their waveforms and in calculated tuning direction and quality between days (due to intermittent equipment noise, estimation error, and actual tuning changes). Preferred directions and R² scaling values were recalculated daily to make use of the current properties of the recorded units. Therefore subjects had to learn a slightly different brain-to-cursor-movement relationship each day. Paired t tests were done between subsequent blocks of brain-controlled movements to look for trends within days that would indicate learning of each new relationship. Results showed subjects initially improved their target hit rate by about 7% from the first to the third block of eight closed-loop movements each day (P<0.002), but improvement leveled off after that.

Coadaptive Algorithm

In the open-versus closed-loop, the subjects demonstrated an ability to take on new, more useful cortical modulation patterns within the first several minutes of practice (i.e. significant improvement from the first to the third block of brain-controlled movements within days). Improvement within each day leveled off after about the third block suggesting that there was a limit to the range of possible modulation patterns the animals could make. The subjects could not fully generate the modulation patterns required by the ‘fixed’ decoding algorithm to make the movements with 100% accuracy.

A more appropriate solution is use of an adaptive decoding algorithm which adjusts to the modulation patterns that the subjects can make. By using an algorithm which tracks changes in the subjects' modulation patterns, the subjects are able to explore new modulation options and discover what patterns they can produce to maximize the amount of useful directional information in their cortical signals.

This ‘coadaptive’ process has other potential benefits as well. In healthy animal studies, the subjects can be trained to make an initial set of arm movements. The cortical activity recorded during those baseline movements is then analyzed and used to define the cortical decoding algorithm. In practice, immobile human patients can't make these initial movements. They can think about or visualize movement, but Lacourse et al. (1999) [22] has shown the EEG patterns produced in spinal cord injury (SCI) patients while visualizing movements are less distinct than those of healthy subjects during normal arm movements. Basing a fixed cortical decoding scheme on these sub-par signals would not take advantage of the brain's full encoding potential.

It is likely that post-SCI cortex can regain a high level of conscious modulation control once the cortex is put back to functional use. Kennedy et al. (2000) have shown immobile human patients can relearn to modulate individual motor cortex cells—even after long periods of disuse. Wu and Kaas (1999) [41] have shown that unused areas of the motor cortex receive invasive remapping from the neighboring functional body parts after an injury. However, functional magnetic resonance imagining (fMRI) studies show that the underlying motor maps are still maintained after SCI, and activity in these areas can still be evoked voluntarily (Shoham et al., 2001) [35].

Having volitional activity in the cortex is critical for neuroprosthetic control. Invasive ‘over-mapping’ from neighboring cortical areas and the lack of kinesthetic feedback may make the initial prosthetic control patterns more abnormal and volatile—at least in the early stages of retraining the cortex. Using a coadaptive algorithm to track changing cortical encoding patterns can enable the patient to work with his current modulation capabilities, allowing him to explore new and better ways of modulating his signals to produce the desired movements. Although the final result may not resemble the original pre-injury signals, the acquired modulation patterns might be better suited for the specific neuroprosthetic control task.

In this coadaptive experiment, the inventors restrained both arms of the monkeys to model the immobile patient. Although the animals were perfectly healthy and did not have cortical changes due to remapping or long-term disuse, the normal activity patterns should have been somewhat altered by the change in kinesthetic feedback. This animal model allowed testing a coadaptive method of evolving a movement decoding algorithm using immobile subjects with altered cortical modulation patterns.

In the open- versus closed-loop experiments, the modified population vector used to translate cortical signals into 3D movements was clearly not optimal. The consistent errors seen in both open- and closed-loop trajectories suggest that more accurate control could be achieved with an improved decoding algorithm which corrected these errors.

The form of a good real-time cortical decoding algorithm needs to be simple and efficient enough for real-time calculation while still deciphering a majority of the information contained within the signals. While it is clear that complex details of the cortical signal can convey additional information about the intended movements of healthy behaving animals (e.g. correlations between units, non-linearities in the tuning functions, etc.), it may not be cost effective to incorporate every possible aspect of the cortical firing patterns into a movement prediction algorithm—especially in the early volatile stages of relearning to use the motor cortex. In this scenario, retraining the cortex to convey information in the most straightforward, easily-decodable form would be ideal. Additional layers of complexity could be added on later once the patient's control skills become more finely tuned.

The most basic and very convenient view of directional coding in the motor cortex is that the units are cosine tuned. Schwartz et al. (1988) [31] showed that this is a fairly good assumption with many cortical units. This assumption makes the math involved with translating cortical activity into a movement trajectory extremely simple and fast. However, most units have slightly narrower tuning functions than a true cosine tuning function.

The open- versus closed-loop experiments, described in the previous chapter, used a population vector which assumed that the neurons were cosine tuned. This over simplification resulted in problems with uneven movement magnitudes and quality throughout the work space (i.e. units could make larger contributions to the movement vector with movements in their preferred directions than 180° away from their preferred direction). By weighting units based on their quality of tuning, the inventors assumed the units provided equal quality of movement information both in and 180° away from their preferred direction. This, however, was usually not the case and may have contributed to the prediction errors seen in the open- versus closed-loop experiments.

This coadaptive experiment, the inventors used a cortical decoding algorithm similar to the population vector except that each unit's contribution to the vector sum was weighted differently with normalized rates above versus below zero. This should allow units to contribute more in directions where they provide more useful information. This still assumes the units encode directional information with a broadly-tuned rate code, and this is a fairly general assumption that seems reasonable to impose on most motor and pre-motor cortex cells.

Equation set 3.1 shows movement calculation using a traditional population vector. PDxi, PDyi, and PDzi represent the X, Y, and Z components of a unit vector in cell i's preferred direction. NRi(t) represents the normalized rate of cell i over time bin t.

Mx(t)=ΣᵢPDxi*NRi(t)

My(t)=ΣᵢPDyi*NRi(t) (3.1)

Mz(t)=Σ₁PDzi*NRi(t)

Equation sets 3.2 and 3.3 show the first step of movement calculation in the coadaptive method. Note the form of Equations 3.1 and 3.2 are similar, but, in Equation 3.2, each unit's weights (Wxi, Wyi, and Wzi) can take on one of two values as specified in Equation set 3.3.

X(t)=ΣᵢWxi*NRi(t)

Y(t)=ΣᵢWyi*NRi(t) (3.2)

Z(t)=ΣᵢWzi*NRi(t)

$$\begin{matrix}Wxi = Wxpi & if NRi ( t ) > 0 \\ = Wxni & if NRi ( t ) < 0 \\ Wyi = Wypi & if NRi ( t ) > 0 \\ = Wyni & if NRi ( t ) < 0 \\ Wzi = Wzpi & if NRi ( t ) > 0 \\ = Wzni & if NRi ( t ) < 0\end{matrix} \qquad ( 3.3 )$$

In this experiment, these positive and negative weights were updated after each complete block of brain-controlled movements to the eight targets. This allowed the weights to follow any changes the animals made in their movement encoding schemes during the day's experiment.

With different positive and negative weights, the expected value of the predicted movement direction would no longer be zero when averaged over movement to all eight targets. For example, if a unit's positive X weight was one and its negative X weight was zero, it would only produce movements in the positive X direction. Therefore, constant X, Y, and Z ‘Expected_Drift’ terms were subtracted out to ensure uniform movements could be made in both positive and negative X, Y, and Z directions. Equation set 3.4 shows this next step in the movement calculation, and details on how the expected drift terms were calculated are presented later on in the text.

mx(t)=Xi(t)−Expected_DriftX(t)

my(t)=Yi(t)−Expected_DriftY(t) (3.4)

mx(t)=Zi(t)−Expected_DriftZ(t)

The average magnitudes of the X, Y, and Z components of the cursor movement were also normalized across components to ensure a uniform scale of movements in all three components. These normalization terms were only adjusted after each complete block of movements to allow for different mean speeds within the block

The process of adjusting the positive and negative weights was designed to identify an effective combination of weights that would enable the subject to make 3D brain-controlled movements using whatever tuning direction and quality the animal's units' took on. Therefore, the weights did not have to match the unit's actual preferred directions. The different components of each unit's positive and negative weights were individually adjusted to redistribute the control as needed throughout the workspace, and to emphasis units when they fired in a range which provided the most useful contributions to the predicted movement.

The weights were adjusted after each block of movements to all eight targets. The positive and negative X, Y, and Z components were independently adjusted for each unit to minimize the prediction error produced by that particular weight and unit during the most recently completed block of movements. Equation sets 3.5 and 3.6 show the changes to each unit's weights needed to reduce the error seen in the previous movement block. This step in the adjustment process evaluates each unit individually as if it were solely responsible for creating the cursor movement.

ΔWxpi=Eₖ[Wxpi(k)*NRi(k)−(Tx(k)−Cx(k))]

ΔWypi=Eₖ[Wypi(k)*NRi(c)−(Ty(k)−Cy(k))] for all Nki(k)>0 (3.5)

ΔWzpi=Eₖ[Wzpi(k)*NRi(k)−(Tz(k)−Cz(k))]

ΔWxni=Eₖ[Wxni(k)*NRi(k)−(Tx(c)−Cx(k))]

ΔWyni=Eₖ[Wyni(k)*NRi(k)−(Ty(k)−Cy(k))] for all NRi(k)<0 (3.6)

ΔWzni=Eₖ[Wzni(k)*NRi(k)−(Tz(k)−Cz(k))]

Equation sets 3.5 and 3.6 show that the movement vector produced by unit i was compared with the movement vector needed at each time step k (movement vector needed=[Txk)−Cx(k), Ty(k)−Cy(k), Tx(k)−Cz(k)] were T is the target position and C is the cursor position at time step k). The change needed in the positive weight vector, [ΔWxpi, ΔWypi, ΔWzpi], was calculated as the average difference between the movement vector produced and the movement vector needed for all time steps in the previous block where the normalized rate went above zero (shown by the expectation operator, Ek[] if NRi(k)>0). The change needed in the negative weight vector was similarly calculated using all time step where the normalized rate went below zero (i.e. NRi(k)<0).

After each block of movements, weights were not simply adjusted by the full ΔWi values described by Equations 3.5 and 3.6. However, the adjustments were proportional to the calculated ΔWi values. Each weight was changed more if its current value produced relatively large but consistent errors, and less if it caused small and inconsistent errors (like one would expect if it was adjusted just right).

If a current weight component produced relatively large and inconsistent errors, the weight component itself would be scaled down relative to the other unit's weights. If the errors were small but consistent, its relative magnitude would be scaled up.

Additional update rules enabled the coadaptive algorithm to search the possible weight space and hold on more strongly to groups of weights which produced the most successful movements. Movement success was defined first by the number of targets hit, and next by how quickly the targets were reached. Because the average movement magnitude at each time bin was held constant, selecting groups of weights based on the shortest movement time was equivalent to selecting weights which produced the straightest, most direct paths to the targets.

Methods

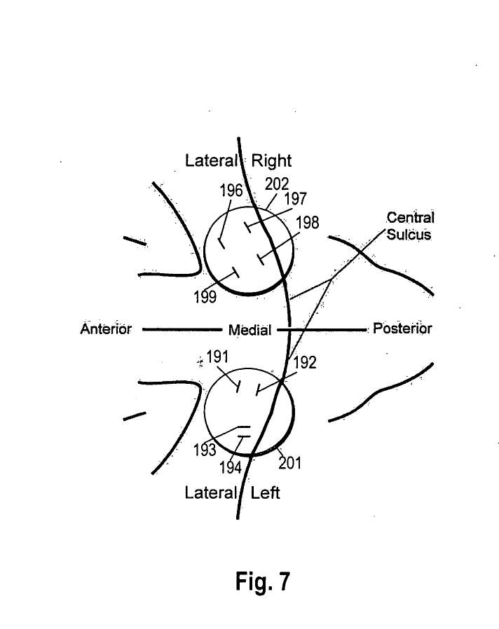

The virtual reality setup used in this experiment was the same as described in the open versus closed-loop experiment. Two healthy macaques were used—monkey ‘M’, whose implants were shown in FIG. 4a (both hemispheres now implanted), and a new monkey, ‘O’. Monkey ‘O’ initially had four 16-microwire arrays 191-194 (FIG. 7) implanted in the left motor and pre-motor areas. Arrays were 2×8 platinum iridium microwires with a Teflon/polyimide coating. That implant's recordings were not very consistent from day to day and disappeared completely after only 20 days of recording. A similar implant 196-199 was done on its right hemisphere, but, again the units were not very stable and only lasted through 12 recording sessions. Passive and active arm manipulation showed subject ‘O’s units were related to both proximal and distal arm movements in both implants. FIG. 7 shows the estimated array locations in subject ‘O’. With this monkey, one large (1.8 cm) craniotomy was made in each hemisphere at 201, 202, and this may have contributed to the difference in recording stability between animals.

In FIG. 7, the electrode placement is in subject ‘O’. The gray areas indicate the craniotomies. The black straight lines show the approximate electrode placements.

At the beginning of each day's experiment, eight to ten minutes of normal center-out hand-controlled right arm movements where collected along with the corresponding neural activity. This data set was used to characterize the cells' baseline tuning behavior under hand-control. Following the free arm movements, both arms were restrained to model the immobile patients. The monkeys then performed the coadaptive brain control task for another 25 to 70 minutes. Either random numbers or the cells' actual preferred directions (determined from that day's hand-controlled movements) were used as initial starting values for both the above- and below-zero sets of X, Y, & Z coefficients (each set first normalized to a unit vector). Because initial performance was so poor in either case, the task started each day with large, easy-to-hit targets (4 cm radius). As coadaptation progressed, the target size was decreased or increased by 1 mm after each complete block of eight targets depending on if the average target hit rate over the last three blocks was above or below 70% respectively. This was done to encourage the development of more directional accuracy as the movement prediction algorithm improved. The target was not allowed to get smaller than 1.2 cm in radius to ensure it would not be obscured by a 1.0 cm radius cursor.

At the end of each day's experiment, tuning function statistics were calculated from cortical data collected during the brain-controlled movements and compared with values obtained from that day's baseline hand-controlled movements. Monkey ‘M’ did these hand and brain-controlled experiments for 39 days of regular practice, and then an additional 11 days of intermittent practice spread over a three month period. After a one and a half month break, regular practice was then resumed. Subject ‘M’s cells remained relatively stable throughout this time. Therefore, the animal's performance statistics were compared across days to look for indications of learning and retention of skills with varying degrees practice.

Monkey ‘O’, however, was newly implanted at the start of these experiments. The units recorded on both left and right hemisphere implants changed considerably from day to day before they disappeared all together. This instability made it difficult to track or train individual cells over time, and, therefore, analysis of day-to-day learning was not done.

The brain-controlled movement task was a ‘fast-slow’ task during subject ‘O’s first implant and during subject ‘M’s 39 days of regular practice and 11 days of intermittent practice. In this version of the experiment, the first 10 to 15 minutes of brain-controlled movements were done with a fast cursor speed and a short movement time (speed ≈100 mm/sec; time allowed=one sec). Then the cursor gain was slowed to one third the speed and the movement time was tripled (speed ≈33 mm/sec; time allowed=three see). This sequence allowed for rapid initial changes in the weights followed by fine tuning of the weights for better directional control.

After subject ‘O’s second implant, and after subject ‘M’s one and a half month break, the task was change to just a ‘fast’ task (speed ≈100 mm/sec; time allowed=one sec). The goal this time was to develop more speed control (i.e. normal velocity profiles and good control over stopping at the desired target).

Once the animals were proficient in the coadaptive task, each days' experiments were ended with a nonadaptive movement task (a Constant Parameter Prediction Algorithm or CPPA task). In this case the weights were held constant and no longer adapted. The subjects were required to use the nonchanging cortical decoding algorithm to make movements from the center-out to the targets and back to the center. Six additional targets were added to the original eight target positions.

Results and Discussion

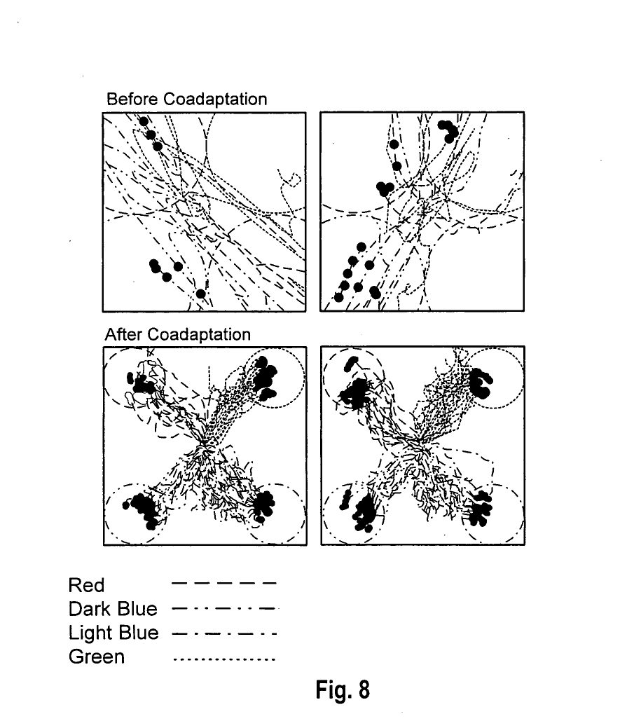

On all days, the initial quality of the brain-controlled movements started out poor. The top two squares in FIG. 8 show an example of center-out trajectories before the algorithm weights changed much from the original preferred direction values used (first two movement blocks, day 39). At this initial stage, there was little organization or separation between trajectories to the different targets. The bottom two squares show examples of trajectories from the same day after about 15 minutes of coadaptation or 36 to 53 updates of the algorithm weights. By that time, the trajectories were well directed and there were clear separations between the groups of trajectories to each of the eight targets.

In FIG. 8, are the trajectories before and after coadaptation for subject ‘M’ on day 39. Movements to the eight 3D targets are split into two plots of four targets for easier two dimensional viewing. Empty circles show the planar projection of the potential target hit area (radius equals the target radius plus cursor radius). Small black filled dots show when the target was actually hit. Trajectories were plotted in the same shade of gray as their corresponding target hit area circles. The upper two squares show the center-out trajectories from the first two blocks of movements before the weights changed much from their initial values. Weights used were either the preferred directions calculated from hand-controlled movements, or one adjustment away of these values. The bottom two squares show center-out trajectories after 15 minutes of coadaptation (after 36 to 53 adjustments of the weights).

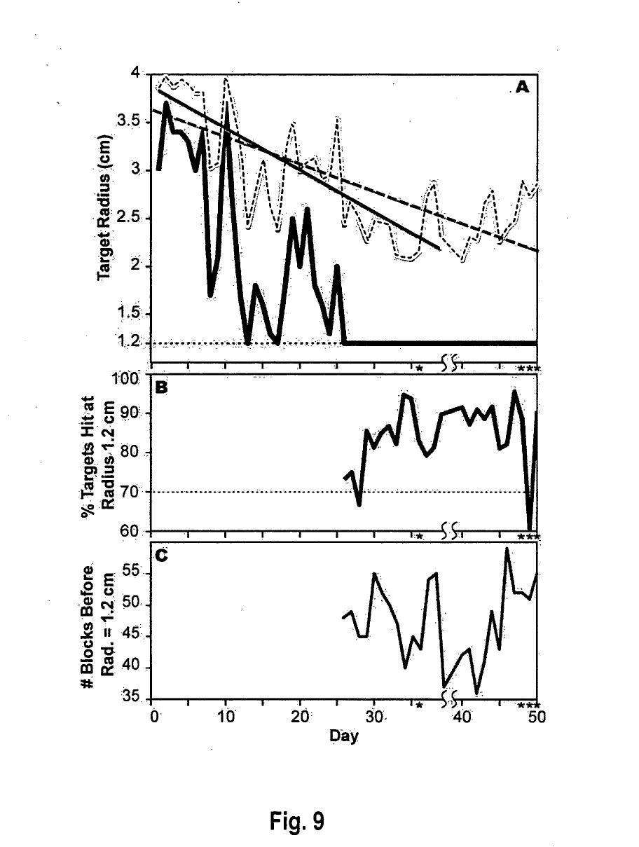

The quality of movements also improved across days as the monkey became more experienced with the coadaptive task. Since the target radius was adjusted to try to maintain a constant target hit rate of 70%, the minimum and mean target radii become measures of how accurate the subjects' movements were. FIG. 9A shows subject ‘M’s minimum (thick black line) and mean (thick gray line) target radii for each day of the fast-slow coadaptive task. On each day, the initial target radius was 4.0 cm and the radius was never reduced below 1.2 cm (black dotted line)—even if the hit rate went above 70%. The actual percent of the targets hit at target radius 1.2 cm is shown in FIG. 9B. This shows that some days' performance improved beyond the 70% hit rate at 1.2 cm target radius. The number of blocks or parameter updates before the target reached 1.2 cm is shown in FIG. 9C.

The break in the ‘Day’ axes indicates when regular coadaptive training was stopped in order to spend time analyzing the data from the first 39 days (left of break). The data to the right of the break is from the eleven days of coadaptive training which were spread over a three-month period after the break. In spite of the intermittent practice during this time, subject ‘M’ was consistently able to get the target radius down to the minimum size (highest performance accuracy level) allowed. The reduction in mean target size appeared to taper off during the last half of the days. However, on days 25-50, additional tasks were preformed after the coadaptive task. Therefore, the coadaptive task was stopped within about 15 minutes or less after the target radius reached its 1.2 cm radius limit. This effectively weakened some of the performance measures on those days, because the ‘mean-target-radius and ‘%-targets-hit’ included fewer good trials after the algorithm converged than it had on earlier days. On days with asterisks, random numbers were used for initial values instead of preferred directions.

FIG. 9 shows performance of subject ‘M’ during regular practice and intermittent practice in the fast-slow coadaptive task. The break between days 39 and 40 marks the end of regular training and the start of intermittent practice. Asterisks indicate days when random numbers instead of preferred directions were used as initial parameter values. A) Minimum (black) and mean (gray) target radii as a function of the number of days of practice. Solid straight gray line is the linear fit of the mean radius for days 1-39, dotted straight gray line is the linear fit of the mean radius to all days. Both slopes are significant at P<10⁻¹⁵. B) Percentage of targets hit during blocks when the target radius was at the lower limit of 1.2 cm. C) Number of blocks of movements or times the weights were updated before the target reached the 1.2 cm lower limit. The one random day within the initial training period and the three random days at the end of the intermittent practice section had similar performance measures to their neighboring days. This suggests that a priori knowledge of the units' tuning functions is not necessary or even beneficial for this algorithm to work.

Toward the end of the intermittent training period, subject ‘M’ took a long time to get the target radius to 1.2 cm (as he did on several occasions during regular training too). Even though the subject could still get the target radius down to 1.2 cm with only intermittent practice, it took longer and longer to do so once regular practice had stopped.

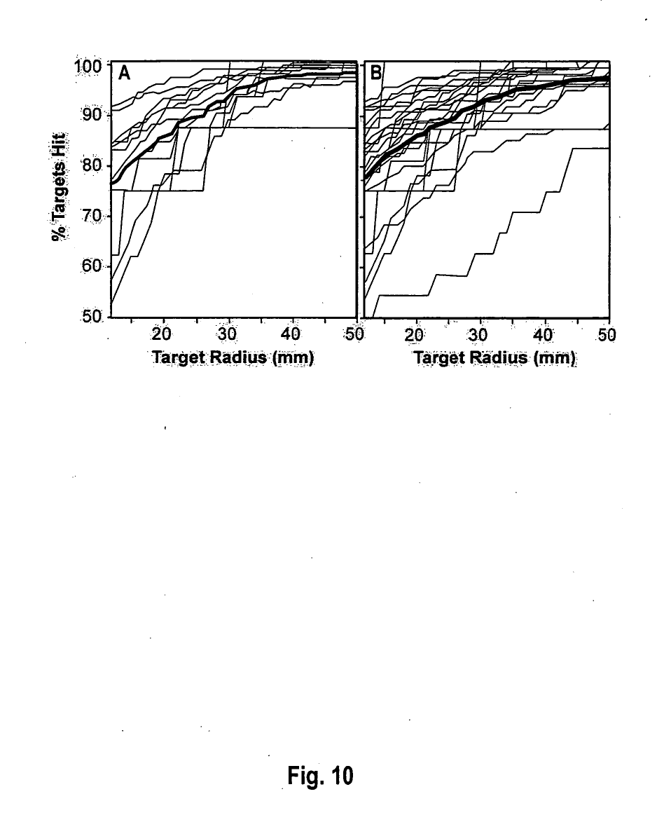

The percentages of targets that would have been hit at larger radii were also calculated from all blocks once the target reached the 1.2 cm lower limit. FIG. 10 shows the daily values (gray) and mean values across days (black) of this calculation. Part A includes only the last 13 days of the regular practice section. Part B also includes the intermittent practice days. Table 1 shows the mean and standard deviation across days of the calculated percent of targets that would have been hit at different radii. The mean percentage of targets hit never reached 100%—even when the target radius was assumed to be 5.0 cm. This is most likely due to the monkey's attention span, and not a problem with its skill level. Large errors in cursor movement often followed loud noises, especially voices, in the neighboring rooms. Also, large errors occurred when the monkeys wiggled in the restraining chair. This often happened after the subjects had been sitting for a long time and had already received a large amount of water. Data contamination from inattentive trials is particularly difficult to avoid in this type of animal experiment because it's impossible to guarantee that the subject is actually trying to hit the target each time.

FIG. 10 shows the percentage of targets that would have been hit had the target been larger. Calculations are for subject ‘M’ and are only from all blocks after the target reach the 1.2 cm size limit. Gray lines show percentage calculations from each day. Black lines are the mean values across days. Calculations were based on A) the final 13 days of the regular training period, and B) all of the final days where the target consistently reached the 1.2 cm lower limit.

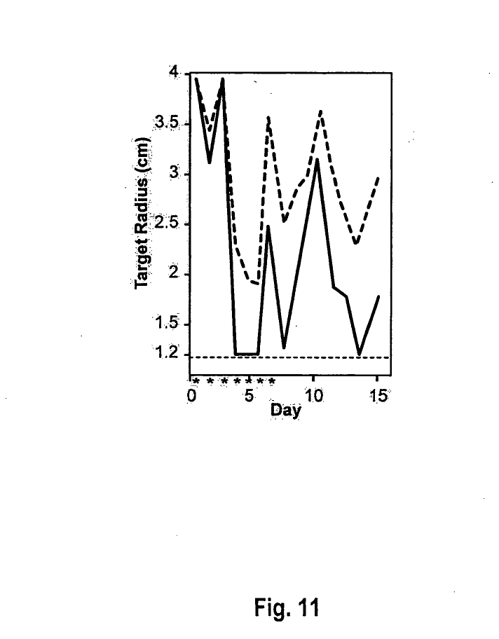

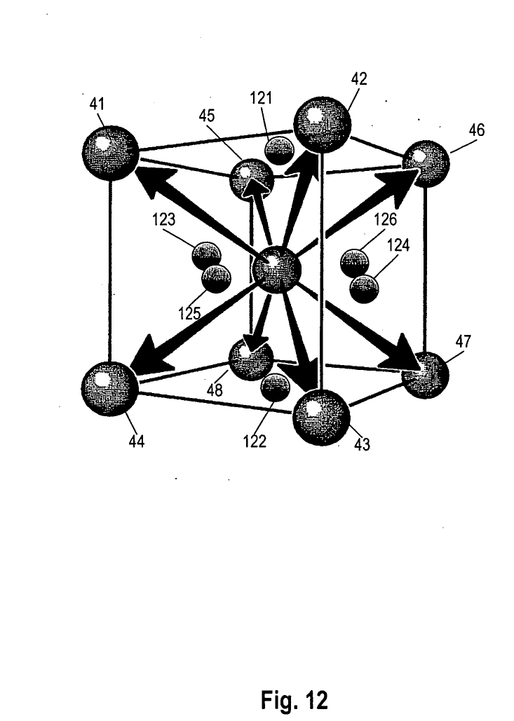

When the next sequence of experiments started after a month and a half break, monkey ‘M’s performance was initially very poor (FIG. 11). The first two days were conducted using the old fast-slow sequence before moving on to the fast-only task on day three. Although monkey ‘M’ was proficient in the fast-slow task months earlier, the subject was now reluctant to do the task and spent much of the time squirming in the chair. On day three, the fast task was started, and by day four, the subject was capable of doing this task at the smallest target size (highest precession level) allowed.

TABLE 1
Percentage of targets that would have been hit had the targets been
larger than they actually were.

| | | Regular training plus |
|---|---|---|
| Target radius | Regular training only | intermittent training |
| 1.2 cm | 76 ± 12 | 78 ± 12 |
| 1.5 cm | 81 ± 10 | 82 ± 11 |
| 2.0 cm | 86 ± 8 | 86 ± 10 |
| 2.5 cm | 90 ± 7 | 89 ± 9 |
| 3.0 cm | 94 ± 4 | 92 ± 8 |
| 3.5 cm | 97 ± 4 | 95 ± 6 |
| 4.0 cm | 98 ± 3 | 96 ± 6 |
| 4.5 cm | 98 ± 3 | 97 ± 4 |
| 5.0 cm | 98 ± 3 | 98 ± 4 |
| # Days in calculations | 13 | 25 |
| # Units recorded | 64 ± 2 | 64 ± 1 |
| # Units used | 39 ± 2 | 38 ± 2 |
| Note. | | |
| All values were calculated from movements that occurred once the target reached 1.2 cm each day, and were from days 26 or later. ‘# units recorded’ includes hash or noisy units whose weights were reduced in the coadaptation process and, therefore, didn't contribute significantly to the cursor movement. ‘# units used’ is the number whose vector sum of their averaged positive and negative | | |
| #weight magnitudes (normalized as they were in the algorithm) equals 95% of the vector sum of the all averaged positive and negative weight magnitudes | | |

On day six, the required target hold time was doubled to further increase the speed control requirements (from 100 msec to 200 msec). The subject still got the target down to the smallest size allowed on that day, but was unable to consistently repeat this on subsequent days. This may be due to several factors: 1) the task was more difficult, 2) faulty headstages adversely affected the quality of the neural recordings—particularly on days seven, ten and eleven, and 3) during this time, the animal was given extra fruit at the end of each day's experiment. The animals were getting, at most, an extra 50 cc of liquid from the fruit, but their response to the sweet fruit was very intense and aggressive—even after they'd had plenty of water. The anticipation of getting treats after the experiment may have affected their concentration. The fruit was stopped on day nine and any anticipation should have subsided after several days.

FIG. 11 shows the performance of subject ‘M’ upon resuming regular practice after a month and a half break. The black solid line shows the daily minimum target size achieved. The gray line shows the daily mean target size achieved. Asterisks indicate days which started with random numbers for initial weight values. Non-asterisk days started with already-adapted weight from earlier days when the performance was good (each unit's weights normalized to unit vectors). The fast-slow coadaptive task was done on days one and two, and the fast-only task was done on the rest of the days. Longer target hold requirements were started on day seven.

Random numbers were used for the initial weights in the coadaptive algorithm on the first seven days after the break. On subsequent days, the initial weights used were the final adapted weights from a recent day were the performance was good. To ensure all units had an equal chance to contribute to the movement initially, each unit's positive and negative weights were first scaled to unit vectors in both the random and pre-adapted cases. Since some of the best and worst days started with random initial weight value, any benefit of using pre-adapted weights is unclear from this study. However, with motivated human patients and noise-free equipment, starting each new training session using the final adapted weights from the previous session still may help speed up the training process.

Testing of Practical Applications

Following the coadaptive algorithm refinement described above, testing of the animals' ability to use the newly evolved directional encoding schemes in more practical applications ensued. The cortical decoding algorithms produced by the coadaptive process were held constant and no longer allowed to adapt. The subjects were then required to make more practical sequences of movements like one would use in everyday life.

The coadaptive process described above was also used to evolve a decoding scheme for the control of a robotic arm. Once an appropriate cortical decoding algorithm was determined, the subject was required to make sequences of robotic arm movements to random positions in space without further adaptation of the algorithm.

Constant Parameter Prediction Algorithm Task

In the coadaptive task, the subjects learned to make fairly accurate center-out movements to eight corners of the workspace. However, for this algorithm to be practical in the real world, patients will want to make more practical brain-controlled movements than just the center-out movements to eight corner targets. They also will need to be able to take the weights evolved during the coadaptive process, and use them in a non-adaptive constant state. The constant parameter prediction algorithm (CPPA) task was designed to test the animal's ability to use the evolved decoding algorithm in a non-adaptive state, and to use it to make a more useful variety of movements. The resulting brain-controlled movements were also more stringently evaluated for ways to improve the quality of movement control in future experiments.

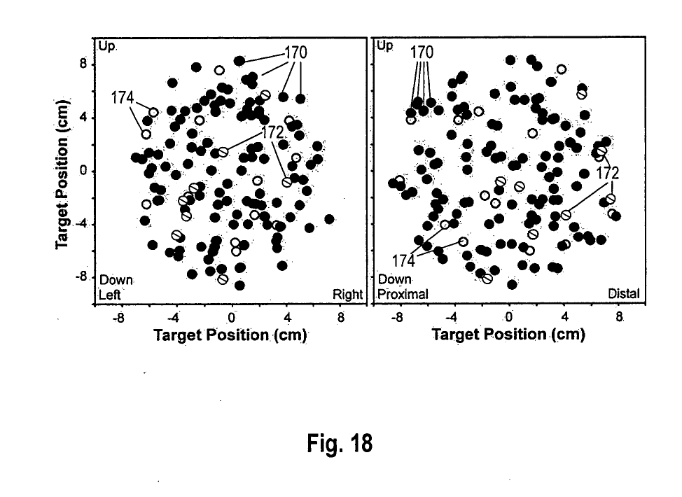

On the last twelve days of subject ‘M’s regular practice period, and the on five of the days of the subject ‘O’s training with the first implant, the subjects performed the constant parameter prediction algorithm or CPPA task. They started the task after completing about 20 minutes to one half hour of the coadaptive task. The weights were held constant during this task and were determined by taking the average of the weights from the coadaptive movement blocks where the performance was good. In this task, as shown in FIG. 18, six ‘novel’ target positions 121-126 were included (straight up 121, down 122, left 123, right 124, proximal 125, and distal 126) in addition to the same eight ‘trained’ targets 41-48 used during the coadaptive task. Instead of just center-out movements, the subjects now had to go from the center to the target and back to the center. This meant the subjects now had to make 180° changes in movement direction—something they had never been required to do before during co-adaptation.

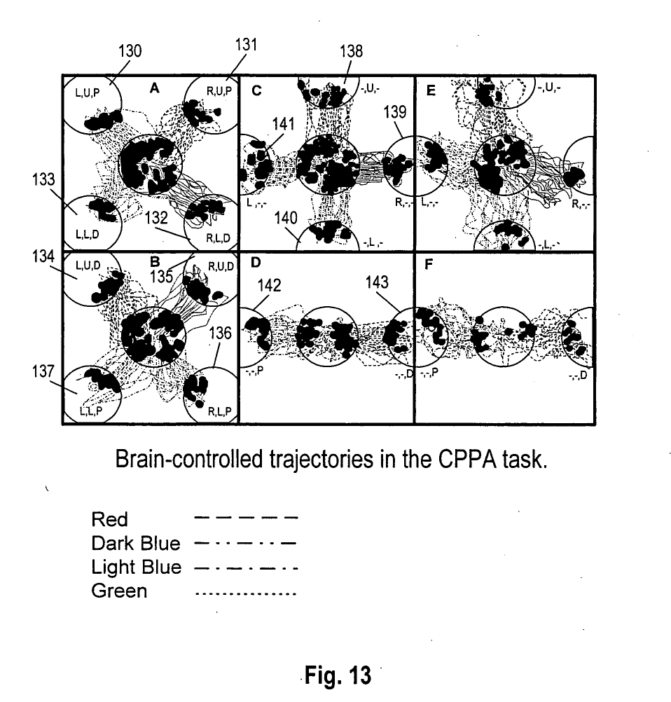

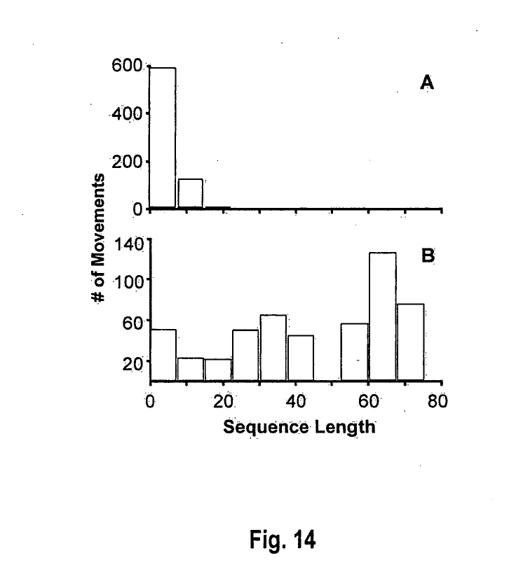

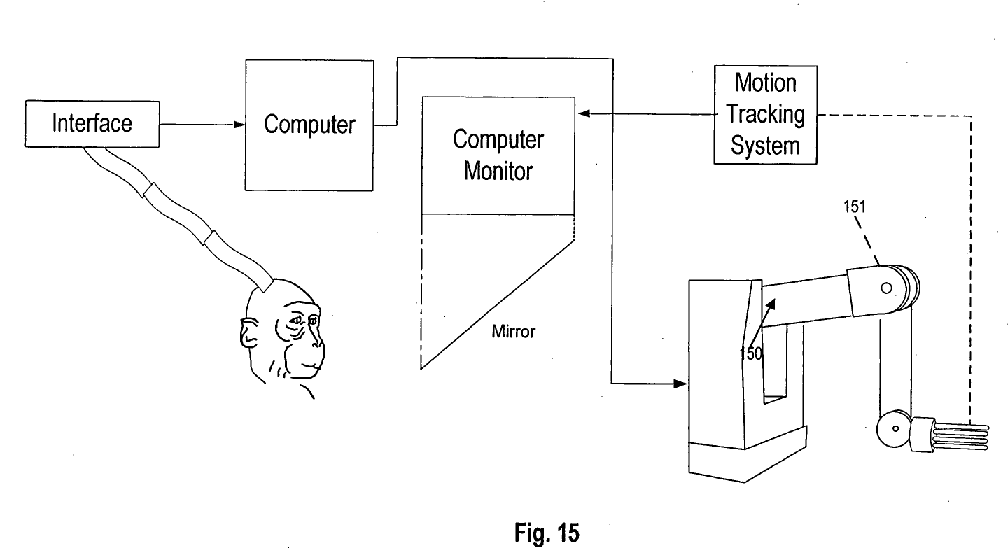

FIG. 13 plots examples of brain-controlled center-to-target-to-center trajectories from this task. Parts A and B show subject ‘M’s trajectories to the eight ‘trained’ targets which were also used in the coadaptive task. Parts C and D show subject ‘M’s trajectories to the six ‘novel’ targets which were not trained for during the coadaptive task. Trajectories are color coded to match their intended targets. The outer circles represent two dimensional projections of the possible target-hit areas (i.e. possible hit area radius equals target radius, 2.0 cm, plus cursor radius 1.2 cm). The radial distance from the center start position to each target center was 8.66 cm. In the task, the cursor started from the exact center, moved to an outer target, then returned to hit the center target (gray center circle shows center target hit area). The black dots indicate when the outer targets or center target was hit. The three letters by each target indicate Left (L)/Right (r), Upper (U)/Lower (L), Proximal (P)/Distal (D) target locations. Dashes indicate a middle position. A—D show trajectories for monkey ‘M.’ A and B are to the eight ‘trained’ targets used in the coadaptive task. C and D are to the six ‘novel’ targets. E and F are novel target trajectories made by monkey ‘O.’

There was very good separation between targets showing the animal had a high level of selective control over the movement direction. The subjects could easily stop and make the required 180° change in movement direction. In most cases, the subject was able to make fairly direct movements taking the shortest path to the targets. Usually there was little curvature in the trajectories, and the trajectories usually went toward the midpoint of the target hit area However, when deviations from a direct path occurred, the deviations were usually consistent to the same targets within each day. Perturbation studies suggest that, with regular practice, subjects will learn to compensate for regular deviations in their trajectories (Gandolfo, Li, Benda, Schioppa & Bizzi, 1999; Thoroughman & Shadmehr, 2000; Weber, 2001). The fact that consistent deviations did not always straighten out with repeated movements suggests that, at times, there was still some minor non-uniformity in the subjects' movement ability over the workspace. If it was just as easy for the animal to move to the center of the target direction as it was to hit the target from the side, one would expect the subject to learn to do so within one experimental session. In human patients, it may be possible for the recorded units to adapt gradually over time and even out the level of control over the workspace. However, a more immediate solution would be to make a corrective skewing function which takes the output of the cortical control algorithm and remaps it throughout the workspace, so the patient can move equally well in all directions using the cortical modulation patterns he is able to produce. These skewing functions could potentially smooth out the minor deviations sometimes seen in this task, but they could not correct for gross inadequacies of the signals due to under-representation of preferred directions in any one orthogonal movement component.

The algorithm was designed to normalize the magnitude of movements between the X, Y, and Z directions by normalizing each component by the estimated magnitudes of the X, Y, and Z movement components from the population sum. This, however, doesn't compensate for correlations between the X, Y, and Z components. For example, if the majority of predicted movements with a positive X component also consistently have a positive (or negative) Y component, then there will be asymmetries in movement gain and control along the diagonal axes even though the average movement magnitudes are still equal in X, Y, and Z. Additional correction terms should be added to the coadaptive algorithm to normalize these correlations and eliminate the difference in gain along the diagonals.

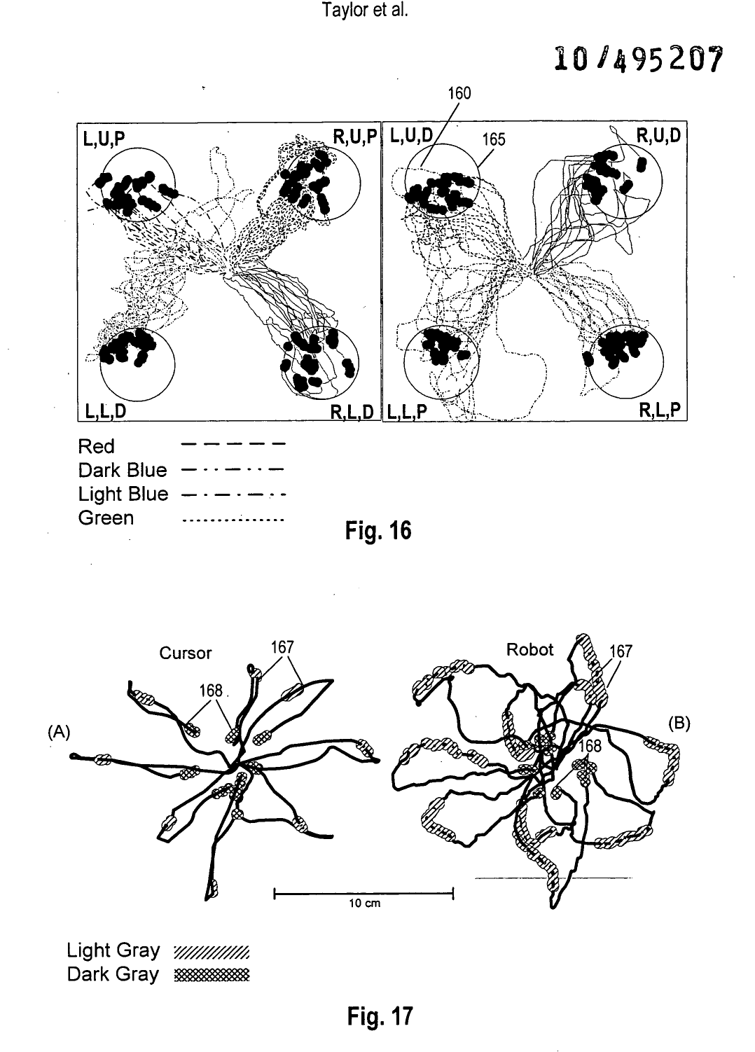

This difference in gain along different diagonal axes resulted in some targets having very smooth trajectories, while others showed more jitter. Note the left upper proximal target and the right, lower, distal targets have smoother trajectories than the ones perpendicular to them (FIG. 16). This is also true when comparing the right upper distal and left lower proximal targets to their perpendicular counterparts (FIG. 16). Movements along the smooth diagonal trajectories had higher gains in those directions than movements to the more jerky orthogonal directions. The jitter in the orthogonal directions stemmed from the subject having a difficult time keeping the high-gain orthogonal movement component at zero. However, the subjects were able to make fairly direct movements to targets in the lower-gain directions by ‘zigzagging’ the high-gain orthogonal component around zero. Additional low-pass filtering could help this problem, although incorporating correlation normalization terms into the algorithm should eliminate the problem altogether.

Parts C and D show subject ‘M’s trajectories to the six ‘novel’ targets which the animal had not trained on during the coadaptive task. These trajectories were of comparable accuracy and smoothness as the ‘trained’ targets in parts A and B. Paired t tests showed there was no significant difference between the novel and trained targets in either the target hit rate (P<0.5) or center-to-target time (P<0.6). There was a slight significant difference in the target-to-center time between the novel and trained targets. The subject actually returned to the center faster from the novel targets than the trained targets (P<0.02). This may be due to the subject's difficulty with moving in certain diagonal directions because of the uncompensated correlations between X, Y, and Z components.

There was a significant difference, however, in the target hit rate to targets requiring proximal versus distal cursor movements (P<10⁻¹⁰). This includes center-to-target movements and target-to-center movements. There were probably several reasons for this. First of all, in proximal movements, the target usually obscured the view of the cursor at some point in the movement. Although the targets were slightly translucent, the details of the double image, needed to correctly perceive depth, were washed out by the target's image imposed over the cursor's image. This points out the importance of including proprioceptive feedback in certain types of neuroprosthetic systems. Although some tasks, like controlling a computer cursor or steering a car, would generally rely on visual feedback in both healthy and neurophrosthetic users, in other tasks, such as using an FES system or a prosthetic limb, the user would benefit from proprioceptive feedback in situations where visual feedback is not practical.

Additionally, subject ‘M’ had an under-representation of units tuned along the X or proximal/distal axis. The units the animal did have were more sharply tuned than the ones with large Y or Z components. This resulted in a larger discrepancy between the positive and negative X weights and a larger X drift term compared to the Y and Z components. Although the drift terms ensured that the subject could make equal magnitude of movements in the positive and negative directions with unequal positive and negative weights, they also caused the cursor to move when the subject was at rest (i.e. when the firing rates were at their mean levels). Therefore, when the monkey was trying to move the cursor proximally, if there was a pause in the effort (such as when the cursor was obscured by a target and the animal was unsure which way to move), the cursor would drift distally.

Requiring effort to remain stationary is not the optimal situation for a neuroprosthetic device. One possible solution would be to add additional selection criteria in the coadaptive task that scales down weights which have large discrepancies between their positive and negative values. The algorithm will then seek out combinations of weights which will not be as dependent on large drift terms. The purpose of having different positive and negative X, Y, and Z weights was to compensate for the difference in the magnitude and quality of rate modulation above versus below the mean in non-cosine tuned units. With regular practice, however, patients will most likely be able to train their units to become cosine tuned. This will eventually alleviate the need for separate positive and negative weights and, therefore, will eliminate the need for the drift terms.

FIG. 16E and F show novel target trajectories made by subject ‘O’ on the fifth and last day the animal did the CPPA task after the first implant. On this day, 31 units were recorded, but most of them were poor-quality noise channels. The weights adapted to make use of 13 of those units. This was the number of units where the magnitude of the vector sum of the averaged positive and negative weight vectors made up 95% of the magnitude of the vector sum of all averaged positive and negative weight vectors. In spite of the low number of useful units, the animal was able to make very selective target-directed movements, although they were not as smooth as subject ‘M’s movements. Part E also shows some slight consistent skewing of the movements, which happened in both animals from time to time.

Subject ‘O’ also had a significantly lower hit rate to proximal targets than distal targets (P<0.005), but had no significant difference between the novel and trained targets in either the target hit rate (P<0.3) or the target-to-center time (P<0.8). The center-to-target time was significantly less in the novel targets than the trained targets (P<0.01). The fact that both animals did as well or better to the novel targets than to the trained targets, suggests the algorithm was able to generalize new movement directions based on data acquired during the eight-target center-out task. Additionally, the subjects' ability to stop and change directions shows the algorithm was also able to generalize to new velocity and sequencing requirements. These results are summarized in Table 2.

TABLE 2
Performance results from the constant parameter prediction
algorithm task

Monkey

| | |
|---|---|
| ‘M’ | ‘O’ |

% Targets hit

| | | |
|---|---|---|
| Novel | 80 ± 26 | 73 ± 29 |
| Trained | 77 ± 24 | 62 ± 30 |
| Center after novel | 80 ± 22 | 72 ± 25 |
| Center after trained | 82 ± 19 | 70 ± 21 |

Average movement time (sec)

| | | |
|---|---|---|
| Novel | 1.5 ± 0.5 | 2.0 ± 0.6 |
| Trained | 1.5 ± 0.6 | 2.6 ± 0.7 |
| Center after novel | 1.3 ± 0.7 | 2.0 ± 1.1 |
| Center after trained | 1.6 ± 0.8 | 2.0 ± 0.9 |

Miscellaneous

| | | |
|---|---|---|
| Number of days in calculations | 12 | 5 |
| Number of units recorded | 64 ± 2 | 31 ± 2 |
| Number of units used^{a} | 38 ± 2 | 17 ± 2 |

Note.
^{a}Number of units where the magnitude of the vector sum of the averaged positive and negative weight vectors made up 95% of the magnitude of the vector sum of all averaged positive and negative weight vectors.

Besides movement generalization, the goal of the CPPA task was to check the viability of using the coadaptive process to determine a brain-control algorithm which could then be used to control a prosthetic device for an extended period of time without requiring further adaptation of the weights. This coadaptive algorithm would have limited practical applications if the brain fluctuated on a time scale that would make the derived weights invalid before they could be put to practical use. However, the true length of time before the weights needed re-calibrating could not be determined. The animals were reward driven, and their willingness to do the task would decline as they became less thirsty. Since the hand-control and coadaptive procedures preceded the CPPA task, the animals were usually not very thirsty by the CPPA task. They would be easily distracted by noises outside the room, and would stop paying attention to the screen. Often, the sound of the reward device would bring their attention back to the task, and the animals would go back to making the same quality of movements as before the distractions.

The subjects generally did the CPPA task for between 15 and 30 minutes before they were no longer thirsty and would just simply stop working. Although this is not a long period of time, the quality of the movements did not decline during this time suggesting the adapted weights remained valid. The consistency of the weights after the first couple minutes of the coadaptive task also implied the cortex remained stable during that time (between 20 and 60 minutes). Finally, the consistency of the preferred directions between days further indicates that once a set of decoding parameters are determined with the coadaptive task, a patient should be able to use those parameters to control their prosthetic device for a useful amount of time.

Table 3 shows how the subjects' performance in the CPPA task changed with daily practice (regression slopes and P values). Both subjects improved their performance in all performance measures across days, although these improvements were not significant in subject ‘O’ with only five days of data ‘Sequence length’ refers to the number of consecutive movements without missing the intended target (center-to-target or target-to-center movements; missed targets have a sequence length of zero).

TABLE 3
Change in CPPA-task performance variables per day,
and its significance.

| Monkey | |
|---|---|
| ‘M’ | ‘O’ |

| | Δ/day | P< | Δ/day | P< |
|---|---|---|---|---|
| % Center-to-target hits | 2.7 | 0.001 | 3.7 | 0.4 |
| % Target-to-center hits | 3.2 | 0.01 | 4.0 | 0.07 |
| Mean sequence length | 2.5 | 0.01 | 1.7 | 0.2 |
| Max sequence length | 4.0 | 0.01 | 4.0 | 0.2 |

FIG. 17 shows the distribution of subjects ‘M’ sequence lengths on the first (A) and last (B) days of the task. Although the monkey took long pauses when distracted, by the last day of practice, the animal was able to make long continuous sequences of movements when attentive.

Although the movements in the CPPA task were not as smooth as normal healthy arm trajectories, they were a significant improvement over what other real-time brain-control algorithms have been able to produce up to this date (Serruya et al., 2002; Meeker et al. 2001; Chapin et al., 1999). Furthermore, the types of problems encountered with the movements should be avoidable or easily corrected by adding correlation correction terms to the algorithm, and remapping any distorted output into a more uniformly-distributed movement space. These subjects also demonstrated that their cortical activity could be used to make long continuous sequences of movements. This level of movement control and flexibility could greatly improve the quality of life for many severely-disable patients.

Brain-Controlled Robot

In the virtual world, the brain-controlled cursor goes exactly where the cortical control algorithm tells it to. The cursor itself has no inertial properties, and it does not add additional variability into the system. However, many neuroprosthetic devices are not so exact. Physical devices, such as wheelchairs, prosthetic limbs, or FES-controlled paralyzed limbs, have distinct inertial properties. The relationships between the command input and the device output may be highly variable due to the system itself being non-deterministic, or due to external perturbations.

Monkey ‘M’s ability to transfer the virtual-cursor control skills to a six-degrees-of-freedom Zebra-Zero robotic arm (designed by Zebra Robotics, Inc.) was tested in both the coadaptive task and a new constant-parameter task. The arm is a full six-axis manipulator with control, using an open architecture PC-based controller. In this experiment, monkey ‘M’s cortical signals controlled the movements of the robotic arm using the same coadaptive 25 algorithm as was used in the virtual cursor task. As illustrated in FIG. 18, although the monkey now controlled the robot directly, the animal still viewed the targets and a brain-controlled cursor 40'through the same virtual reality set up as in the previous experiments.

This time, however, the cursor movements were determined by the real-time position of the brain-controlled robot 150. Optotrak® position markers 51 were placed on the end of the robot arm, and the robot's position controlled the position of the virtual cursor. This way, the task was still familiar to the subject. However, the dynamics of the cursor now were different. The cursor movements now showed the lag, jitter and movement inaccuracies of the robotic arm.

In the coadaptive robotic task, the lower limit on the target size was set to 1.5 cm. The subject was able to reach and maintain this level of accuracy after the first few days of practice with the robot. Trajectories from the coadaptive task are shown in FIG. 16. The circles show two dimensional projections of the possible target hit area and are color coded to match their trajectories. Black dots indicate when the target was successfully hit.

As in the virtual coadaptive task without the robot, there was good separation between trajectories to the different targets. Movements to most targets were as good or almost as good as in the virtual coadaptive task. The subject had problems getting the robot to one area of the workspace, however, more so than other areas. This may have been due to inadequacies in the cortical control signal, due to the robot having difficulty executing movements in that area of the work space, or a combination of the two. Although the trajectories would occasionally go quite far in the wrong direction, the subject was usually able to redirect the robot back to its intended target (e.g. see the cyan trajectory 160 at the magenta target 165 in the right panel of FIG. 16).

The coadaptive task was then followed by a new version of the constant parameter prediction algorithm (CPPA) task which also used the robot. This version still required the subject to move back to the center after hitting the target, but the targets were now in random positions and at random radial distances. FIG. 17 shows two dimensional projections of sample trajectories from the non-robotic (A) and the robotic (3) CPPA tasks. Light gray dots 167 indicate when an outer target was hit, and the darker grey dots 168 show when the trajectories returned and hit the center target.

In the virtual center-target-center task, the animal could easily make 180° changes in the cursor movements. However in the robot task, the inertia of the robot makes this rapid change in direction difficult to execute. Instead, the animal took on a strategy of ‘looping’ through the targets, making do with the limited ability of the motors to decelerate the robot. FIG. 18 shows target positions from the first day subject ‘M’ did the CPPA task with the robot. Black dots 170 indicate targets positions for movements that successfully hit the target and returned to the center.- Gray dots 172 indicate target positions that were hit, but the robot did not return to the center. Empty circles 174 show target positions which were not hit. The data in FIG. 18 was recorded after only one half hour of practice in the robot center-target-center task. In spite of the more limited movement abilities of the robot, the subject was able to hit the targets and return to the center a majority of the time.

Within a very short period of time, the subject learned to work within the limitations imposed by the dynamics of a physical brain-controlled system. It is likely that human patients will also adjust easily to a wide variety of physical devices. However, in this experiment, the inventors co-adapted the brain-control algorithm using brain-controlled movements of the specific device. This strategy may have benefits over co-adapting a brain-control algorithm in a virtual environment and then applying the algorithm to control physical devices. By adapting the algorithm weights to the imperfect movements of the device, the weights may evolve to minimize the effect of some of these imperfections.

Although preferred embodiments of the invention have been described in detail, it will be readily appreciated by those skilled in the art that further modifications, alterations and additions to the invention embodiments disclosed may be made without departure from the spirit and scope of the invention as set forth in the appended claims. For example, although the firing rate is used in the specific, exemplary preferred embodiment, other characteristics of electrical impulses may be used with the methods described and with the system described. The coadaptive algorithm and control functions arrived at using that algorithm are useful in other control applications than those specifically described.

### References

[1] Craggs, M. D. (1975). Cortical control of motor prostheses: using the cord-transected baboon as the primate model for human paraplegia. Advances in Neurology, 10, 91-101.

[2] Wolpaw, J. R., Birbaumer, N., Heetderks, W. J., McFarland, D. J., Peckham, P. H., Schalk, G., Donchin, E., Quatrano, L. A., Robinson, C. J., & Vaughan, T. M. (2000). Brain-computer interface technology: a review of the first international meeting. IEEE Transactions on rehabilitation engineering, 8, 164-173.

[3] Chapin, J. K., Moxon, K. A., Markowitz, R. S., & Nocolelis, M. A. L. (1999). Real-time control of a robot arm using simultaneously recorded neurons in the motor cortex. Nature Neuroscience, 2, 664-670.

[4] Wessberg, J., Stambaugh, C. R., Kralik, J. D., Beck, P. D., Laubach, M., Chapin, J. K., Kim, J., Biggs, S. J., Srinivasan, M. A., & Nicolelis, M. A. L. (2000). Real-time prediction of hand trajectory by ensembles of cortical neurons in primates. Nature, 408, 361-365.

[5] Georgopoulos, A. P. Kettner, R. E., & Schwartz, A. B. (1988). Primate motor cortex and free arm movements to visual targets in three-dimensional space II: coding t: direction of movement by a neural population. The Journal of Neuroscience, 8, 2'2937.

[6] Abbott, L. F., & Salinas, E. (1994). Vector reconstruction from firing rates. Journal of Computational Neuroscience, 1, 89-107.

[7] Lee, D., Port, N. L., Kruse, W., & Georgopoulos, A. P. (1998a). Neural population coding: multielectrode recordings in primate cerebral cortex. In Eichenbaum, H., & Davis, J. L. (Ed.), Neuronal ensembles: strategies for recording and decoding (pp. 117-136). New York: Whiley.

[8] Abeles, M. (1991). The probability for synaptic contact between neurons in the cortex. In Corticontics: neural circuits and the cerebral cortex. (pp. 65-91). Cambridge, Mass.: Cambridge University Press.

[9] Ashe J. (1997). Force and the motor cortex. Behavioral Brain Research, 86, 1-15.

[10] Caminiti, R., Johnson, P. B., & Urbano, A. (1990). Making arm movements within different parts of space: dynamic aspects in the primary motor cortex. Journal of Neuroscience, 10, 2039-2058.

[11] Evarts, E. V. (1968). Relation of pyramidal tract activity to force exerted during voluntary movement. Journal of Neurophysiology, 31, 14-17.

[12] Fetz, B. E., & Finocchio, P. D. (1975). Correlations between activity of motor cortex cells and arm muscles during operantly conditioned response patterns. Experimental Brain Research, 23, 217-240.

[13] Fitts, P. M. (1954). The information capacity of the human motor system in controlling the amplitude of movement. Journal of Experimental Psychology, 47, 381-391.

[14] Pu, Q, G., Flament, D., Colz, J. D., & Ebner, T. J. (1995). Temporal coding of movement kinematics in the discharge of primate primary motor and premotor neurons. Journal of Neurophysiology, 73, 835-854.

[15] Gandolfo, F., Li, C.-S. R., Benda, B. J., Padoa Schioppa, C. & Bizzi, E. (1999). Cortical correlates of learning in monkeys adapting to a new dynamical environment. Proceedings of the National Academy of Sciences USA, 97, 2259-2263.

[16] Ghez, C. (1991a). Voluntary movement. In E. R. Kandel, J. H. Schwartz, & T. M. Jessell (Ed.), Principles of neural science (3^{rd} ed.) (pp. 611-613). Norwalk, CN: Appleton & Lange.

[17] Ghez, C. (1991b). Control of movement. In E. R. Kandel, J. H. Schwartz, & T. M. Jessell (Ed.), Principles of neural science (3^{rd} ed.) (pp535-538). Norwalk, CN: Appleton & Lange.:

[18] Hatsopoulos, N. G., Ojakangas, C. L, Paninski, L. & Donogue, J. P. (1998). Information about movement direction obtained from synchronous activity of motor cortical neurons. Proceedings of the National Academy of Science, 95, 15706-15711.

[19] Isaacs, R. E., Weber, D. J., & Schwartz, A. B. (2000). Work toward real-time control of a cortical neural prosthesis. IEEE Transactions on Rehabilitation Engineering, 8, 196-198.

[20] Kennedy, P. R., Bakay, A. E., Moore, M. M., Adams, K., & Goldwaithe, J. (2000). Direct control of a computer from the human central nervous system. IEEE Transactions on Rehabilitation Engineering, 8, 198-198-202.

[21] Kettner, R. E., Schwartz, A. B. & Georgopoulos, A. P. (1988). Primate motor cortex and free arm movements to visual targets in three-dimensional space III: positional gradients and population coding of movement direction from various movement origins. The Journal of Neuroscience, 8, 2938-2947.

[22] Lacourse, M. G., Cohen, M. J., Lawrence, K. E., & Romero, D. H. (1999). Cortical potentials during imagined movements in individuals with chronic spinal cord injuries. Behavioral Brain Research, 104, 73-88.

[23] Lee, D., Port, N. L., Kruse, W., & Georgopoulos, A. P. (1998b). Variability and correlated noise in the discharge of neurons in motor and parietal areas of the primate cortex. The Journal of Neuroscience, 18, 1161-1170.

[24] Maynard, E. M., Hatsopoulos, N. G., Ojakangas, C. L., Acuna, B. D., Sanes, J. N., Normann, R. A. & Donoghue, J. P. (1999). Neuronal interactions improve cortical population coding of movement direction. The Journal of Neuroscience, 19, 8083-8093.

[25] Meeker, D., Shenoy, K. V., Cao, S., Pesaran, B., Scherberger, H., Jarvis, M., Buneo, C. A., Batista, A. P., Kureshi, S. A., Mitra, P. P., Burdick, J. W., & Andersen, R. A. (2001, November). Cognative control signals for prosthetic systems. Poster session presented at the Society for Neuroscience 31^{st} Annual Conference, San Diego, Calif.

[26] Moran, D. W., & Schwatrz, A. B. (1999). Motor cortical representation of speed and direction during reaching. Journal of Neurophysiology, 82, 2676-2692.

[27] National Spinal Cord Injury Statistical Center. (2001, May). Spinal cord injury facts and figures at a glance. Retrieved June 17, 2002, from http://www.spinalcord.uab.edu/show.asp?durki=21446

[28] Riehle, A., Grun, S., Diesmann M., & Aertsen, A. (1997). Spike synchronization and rate modulation differentially involved in motor cortical function. Science, 278, 1950-1953.

[29] Rieke, F., Warland, D., de Ruyter van Steveninck, R., & Bialek, W. (1997). Models of firing statistics. In T. J. Sejnowski, & T. A. Poggio (Eds.), Spikes. Exploring the neural code (pp 49-54). Cambridge, Mass.; MIT Press.

[30] Serruya, M. D., Hatsopoulos, N. G., Paninsli, L., Fellows, M. R., & Donoghue, J. P. (2002). Instant neural control of a movement signal. Nature, 416, 141-142.

[31] Schwartz, A. B., Kettner, R. E., & Georgopoulos, A. P. (1988). Primate motor cortex and free arm movements to visual targets in three-dimensional space I: relations between single-cell discharge and direction of movement. The Journal of Neuroscience, 8, 2913-2927.

[32] Schmidt, E. M., Bak, M. J., McIntosh, J. S., & Thomas, J.S. (1977). Operant conditioning of firing patterns in monkey cortical neurons. Experimental Neurology, 54, 467-477.

[33] Scott, S. H., & Kalaska, J. F., (1995). Changes in motor cortex activity during reaching movements with similar hand paths but different arm postures. Journal of Neurophysiology, 73, 2563-2567.

[34] Shadmeher, R.& Mussa-Ivaldi, F. A. (1994). Adaptive representation of dynamics during learning of a motor task. The Journal of Neuroscience, 14, 3208-3224.

[35] Shoham, S., Halgren, E., Maynard, E., & Normann, R. A. (2001). Motor-cortical activity in tetraplegics. Nature, 413, 793.

[36] Thoroughman, K. A., & Shadmehr, R. (2000). Learning of action through adaptive combination of motor primitives. Nature, 407, 742-747.

[37] Vaadia, E., Haahnan, I., Abeles, M., Bergman, H., Prut, Y., Slovin, H., & Aertsen, A. (1995). Dynamics of neuronal interactions in monkey cortex in relation to behavioral events. Nature, 373, 515-518.

[38] Watson, G. S., (1956). A test for randomness of directions. Monthly Notices of the Royal Astronomical Society, Geophysics Supplement, 7, 160-161.

[39] Weber, D. J. (2001). Chronic, multi-electrode recordings of cortical activity during adaptation to repeated perturbations of reaching. Unpublished doctoral dissertation, Arizona State University, Tempe Ariz.

[40] Williams, J. C., Rennaker, R. L., & Kipke, D. R (1999). Stability of chronic multichannel neural recordings: Implications for a long-term interface. Neurocomputinig, 26, 1069-1076.

[41] Wu, C. W. H., & Kaas, J. (1999). Reorganization in primary motor cortex of primates with long-standing therapeutic amputations. The Journal of Neuroscience, 19, 7679-7697.

We claim:

1. A method of developing electrical control signals from physiological electrical activity of a human or animal subject comprising:
(a) providing a computational processor,
(b) repeatedly detecting physiological electrical impulses from at least one electrical impulse producing units in or on the subject,
(c) repeatedly supplying to the computational processor electrical representations of one or more characteristics of the electrical impulses,
(d) repeatedly, using the computational processor, calculating, from the electrical representations, movements of at least one physical or computer generated movable object in at least one dimension based on an algorithm programmed in the computational processor,
(e) repeatedly moving the at least one movable object by the calculated amount in a manner discernable to the subject, and
(f) repeatedly modifying one or more terms of the algorithm to enhance movements of the at least one movable object approaching a predetermined movement in response to further detected electrical impulses.

2. The method according to claim 1, wherein step (d) comprises applying the algorithm to representations of one or more characteristics of the detected electrical activity of each of the units, and step (f) comprises modifying one or more terms of the algorithm as applied to the electrical representations corresponding to each unit the electrical impulses of which contribute to the predetermined movement.

3. The method according to claim 1, wherein step (d) comprises calculating an amount of movement as a function of a firing rate of one or more of the units.

4. Thee method according to claim 3, wherein the one or more characteristics of the detected electrical impulses comprise the firing rate, and wherein step (d) comprises:
(i) for each unit calculating a normalized firing rate, NRᵢ(t), in a time window,
(ii) weighting a firing rate-related value for the firing rates of one or more units by their own first positive weighting factor if NRᵢ(t) is greater than zero,
(iii) weighting a firing rate-related value for the firing rates of one or more units negative weighting factor if NRᵢ(t) is less than zero,
and step (e) comprises moving the at least one moveable object a distance dependent upon at least a portion of the weighted firing rate-related values.

5. The method according to claim 2, wherein the at least one moveable object is chosen from a group consisting of real objects and virtual objects.

6. The method according to claim 1, wherein the electrical impulse-producing units are in the subject's cerebral cortex, step (b) comprises implanting at least one array of electrodes in the cerebral cortex of the subject, and step (c) comprises communicating cortex-generated electrical signals via a communication link to the computational processor.

7. The method according to claim 6, wherein implanting at least one array comprises implanting the at least one array in the cerebral cortex of the pre-motor or motor regions of the brain of the subject.

8. The method according to claim 1, wherein step (e) comprises moving the at least one object in the visual field of the subject.

9. The method according to claim 8, wherein the at least one object includes a movable computer display object, and step (e) comprises moving the computer display object in a computer display environment in the visual field of the subject.

10. The method according to claim 10, further comprising providing the subject with a reward upon achievement of a predetermined goal movement.

11. The method according to claim 4, wherein step (f) comprises an iterative updating procedure for adjusting the positive or negative weighting factors from initial values.

12. The method according to claim 11, wherein the initial value is an arbitrarily chosen value.

13. The method according to claim 1, wherein the number of units is less than 100.

14. The method according to claim 1, further comprising:
(g) using the algorithm as modified repeatedly in step (f), without further modification, to translate the electrical signals into control signals for application to a controlled device.

15. The method according to claim 1, wherein the electrical impulse producing units are located in regions selected from the group consisting of the nervous system and the musculature.

16. An electrical controller comprising a computational processor programmed to operate as defined in any one of claims 1-15.

17. Programming for a computational processor having routines for effecting the method of any one of claims 1-15.

18. A control system including an input for receiving physiologically generated electrical impulses, an output representative of direction and distance, a computational processor electrically connected between the input and the output for deriving the output from input physiologically generated electrical impulses, the computational process being programmed with co-adaptively revisable control algorithm adapted to be revised with revisions in input electrical impulses from a test subject.

19. A method of controlling at least one physical or computer generated movable object comprising:
(a) detecting in an animal or human subject electrical impulses caused by electrical activity of units of one or more neurons,
(b) using a coadaption control algorithm that revises with changes in the electrical impulses, deriving from the detected electrical impulses an output representative of direction and distance, and
(c) moving the at least one object in the direction and over the distance represented by the output substantially concurrent with the detection of the impulses.

20. The method according to claim 19, wherein step (b) includes displaying to the subject the movements of step (c).

21. A method of controlling at least one physical or computer generated movable object comprising:
(a) detecting in an animal or human subject electrical impulses caused by electrical activity of units of one or more neurons,
(b) deriving from the detected electrical impulses an output representative of direction and distance,
(c) moving the at least one object in the direction and over the distance represented by the output substantially concurrent with the detection of the impulses; and
(d) the step of deriving further comprising:
(i) displaying to the subject the movements of step (c);
(ii) applying to a computational processor inputs representative of the detected electrical impulses, and
(iii) with the computational processor applying a coadaptive algorithm calculating the direction and distance to be represented in the derived output signal, said coadaptive algorithm having terms varying with the success or failure of the derived output signal moving the at least one object in a predetermined direction.

22. The method according to claim 21, wherein step (b) further comprises deriving the output signal from a detected firing rate of the one or more neurons.

23. The method according to claim 21, wherein step (b) further comprises providing a computer program having the algorithm for converting the inputs representative of the detected electrical impulses to the output signal representative of direction and distance.

24. The method according to claim 21, wherein step (b) further comprises providing a computer program having the algorithm for converting the inputs representative of the detected electrical impulses to the output signal representative of direction and distance.

25. The method according to claim 24, wherein the algorithm includes at least one weighting factor applied to translate physiologically-generated electrical signals into movement direction and distance.

26. The method according to claim 19, wherein step (b) further includes displaying a target to which the object is to move.

27. The method according to claim 25, wherein step (b) further includes displaying a target to which the object is to move, wherein the at least one weighting factor includes a positive weighting factor and step (b) further comprising using the positive weighting factor when the normalized input signal is above zero.

28. The method according to claim 25, wherein step (b) further includes displaying a target to which the object is to move, wherein the at least one weighting factor includes a negative weighting factor, and step (b) further comprising using the negative weighting factor when the normalized input signal is below zero.

29. The method according to claim 25, wherein step (b) includes displaying a target to which the object is to move, wherein the at least one weighting factor includes a positive weighting factor and a negative weighting factor, and step (b) further comprising using the positive weighting factor when the normalized input signal is above zero and using the negative weighting factor when the normalized input signal is below zero.

30. The method according to claim 26, further comprising rewarding the subject upon the object reaching the target.

31. The method according to claim 21, step (a) further comprising implanting a plurality of electrodes in the region of the subject's cerebral cortex and transmitting electrical impulses detected from any electrode detecting the impulses to the computational processor.

32. The method according to claim 20, wherein step (b) comprises applying the algorithm in the coadaptive process during which the subject learns to control movement of the object and the derivation of an output signal representative of direction and distance is dependent on the subject's cerebral cortex region's neuron electrical activity.

33. The method according to claim 22, wherein step (b) further comprises calculating the object's movement on one (x) axis at time t as:
Σᵢ Wx(n_or_{—}p)i*NRᵢ(t)=X(t)
(a) where the index, i, refers to each of a plurality of electrical input signals derived from detected electrical impulses and the values are summed over all signals being used,
(b) NRᵢ(t) is the normalized input signal,
(c) Wxni is the negative weighting factor used if NRᵢ(t)<0, and Wxpi is the positive weighting factor used if NRᵢ(t)>0.

34. The method according to claim 33, wherein step (b) further comprises normalizing each input signal by subtracting its mean and dividing by a constant times its standard deviation to arrive at NRᵢ(t).

35. The method according to claim 33, wherein step (b) further comprises correction of x(t) for drift including calculating the predicted movement in x at time t:
mx(t)=X(t)−Drift(t),
where Drift(t) is estimated as Σᵢ (Wxpi−Wxni)*Eₖ[|NRᵢ(k)|]/2, where Eₖ[|NRᵢ(k)|] is the expected value of |NRᵢ(k)|, the absolute value of input signal i's normalized value.

36. The method according to claim 33, wherein step (b) further comprises correction of calculated movement on one (x) axis at time t, Xot), for drift including calculating the predicted movement in x at time t:
mx(t)=X(t)−Drift(t),
where Drift(t) is estimated as Σᵢ(Wxpi−Wxni)*Eₖ[|NRᵢ(k)|]/2, where Eₖ[|NRᵢ(k)|] is the expected value of |NRᵢ(k)|, which is the absolute value of a normalized value of an input signal i derived from detected electrical impulses.

37. The method according to claim 36, wherein:
ΣᵢWx(n_or_{—}p)i*NRᵢ(t)=X(t)
(a) where the index, i, refers to each of a plurality of electrical input signals derived from detected electrical impulses and the values are summed over all signals being used,
(b) NRᵢ(t) is the normalized input signal,
(c) Wxni is the negative weighting factor used if NRᵢ(t)<0, and Wxpi is the positive weighting factor used if NRᵢ(t)>0.

38. The method according to claim 35, wherein step (b) further comprises calculating Eₖ[|NRᵢ(k)|] from the normalized input signals observed in a most recent complete block of object movements based on one or more detected electrical input signals indicated by the index, i.

39. The method according to claim 35, wherein step (b) further comprises calculating Eₖ[|NRᵢ(k)|], in a noncoadaptive process, by averaging |NRᵢ(k)| over a recent interval and updating this value regularly.

40. The method according to claim 35, further comprising normalizing the magnitude of movement in dimension x at each time t to an expected value of one then scaling by a desired velocity scale (Vscale) to achieve movements of the desired scale Mx(t):
Mx(t)=Vscale*mx(t)Eₖ[|mx(k)|]
where Ek [|mx(k)|] is the expected value of the absolute value of mx(k) taken over all calculation times, k.

41. The method according to claim 40, further comprising calculating Eₖ[|mx(k)|] from the calculate mx(t) from a most recent complete block of object movements.

42. The method according to claim 40, further comprising calculating Eₖ[|mx(k)|] from the calculate mx(t) from a noncoadaptive process, by averaging |mx(k)| over a recent interval and updating this value regularly.

43. The method according to claim 35, wherein step (b) further comprises calculating the at least one object's movement in at least two further dimensions (n1 . . . nx) at a time t as a function of the form:
ΣᵢWₙ_{_{1,2 . . . x}}(n_or_{—}p)i*NRᵢ(t)=N1,2 . . . x(t)
where Wₙ_{_{1,2 . . . x}} ni are the negative weighting factors for unit i's movements in the n_{1,2 . . . x} dimensions, used when unit i's normalized firing rate, NRᵢ(t), is below zero, and Wₙ_{_{1,2 . . . x}} pi are the positive weighting factors for unit i's movements in the n_{1,2 . . . x} dimensions, used when unit i's normalized firing rate, NRᵢ(t), is above zero.

44. The method according to claim 35, wherein one or more additional dimensions of control are simultaneously calculated by the same method, using a new set of positive and negative weights for each additional dimension.

45. The method according to claim 37, wherein one or more additional dimensions of control are simultaneously calculated by the same method, using a new set of positive and negative weights for each additional dimension and additional drift terms are used for each additional dimension of control the additional drift terms being calculated using individual positive and negative weights for each dimension.

46. The method according to claim 45, wherein calculating Eₖ[|NRᵢ(k)|] in a coadaptive process from normalized input signals observed in a most recent complete block of object movements based on one or more detected electrical input signals indicated by the index, i.

47. The method according to claim 45, further comprising calculating Eₖ[|NRᵢ(k)|], in a noncoadaptive process, by averaging |NRᵢ(k)| over a recent interval and updating this value regularly.

48. The method according to claim 45, further comprising normalizing magnitudes of movement in time t of movement dimension, m(t), in each dimension by applying M(t)=Vscale*m(t)/Eₖ[|m(k)|] to each additional movement dimension m (t), where Ek[|m(k)|] is the expected value of the absolute value of m(k) taken over all calculation times, i, for that particular dimension.

49. The method according to claim 48, further comprising calculating Ek[|m(k)|] in a coadaptive process from the movements m(k) calculated in a most recent complete block of object movements.

50. The method according to claim 48, further comprising calculating Ek[|m(k)|], in a noncoadaptive process, by averaging m(k) calculated over a recent interval and updating this value regularly.

51. The method according to claim 19, wherein, in step (b) at least two dimensions of the object's movements are calculated on one (x) and another (y) axis at time t as:
ΣᵢWx(n_or_{—}p)i*NRᵢ(t)=X(t), and
ΣᵢWy(n_or_{—}p)i*NRᵢ(t)=Y(t)
(i) where the index, i, refers to each of a plurality of electrical input signals derived from detected electrical impulses, and the values are summed over all signals being used,
(ii) NRᵢ(t) is the normalized input signal,
(iii) Wxni is the x axis negative weighting factor used if NRᵢ(t)<0, and Wxpi is the x axis positive weighting factor used if NRᵢ(t)>0,
(iv) Wyni is the y axis negative weighting factor used if NRᵢ(t)<0, and Wypi is the y axis positive weighting factor used if NRᵢ(t)>0.

52. The method according to claim 51, comprising correction of X(t) and Y(t) for drift including calculating the predicted movement in x at time t:
mx(t)=X(t)−Drift(t)
and
my(t)=Y(t)−Drift(t),
where
(i) Drift(t) in the X axis is estimated as Σᵢ (Wxpi−Wxni)*Eₖ[|NRᵢ(k)|]/2,
(ii) Drift(t) in the Y axis is estimated as Σᵢ (Wypi−Wyni)*Eₖ[|NRᵢ(k)|]/2,
(iii) Eₖ[|NRᵢ(k)|] is the expected value of |NRᵢ(k)| the absolute value of input signal i's normalized value.

53. The method according to claim 52, wherein step (b) further comprises calculating Eₖ[|NRi(k)|] from the normalized input signals observed in a most recent complete block of object movements based on one or more detected electrical input signals indicated by the index, i.

54. The method according to claim 52, wherein step (b) further comprises calculating Eₖ[|NRi(k)|], in a noncoadaptive process, by averaging |NRi(k)| over a recent interval and updating this value regularly.

55. The method according to claim 52, further comprising normalizing magnitudes of movement in time t of movement dimension m(t), in each of at least two dimensions (x and y) by applying:
Mx(t)=Vscale*mx(t)/Eₖ[|mx(k)|],
and
My(t)=Vscale*my(t)/Eₖ[|my(k)|],
where Ek[|mx(k)|] is the expected value of the absolute value of mx(k) taken over all calculation times, k, for the x dimension, and Ek[|my(k)|] is the expected value of the absolute value of my(k) taken over all calculation times, k, for the y dimension.

56. The method according to claim 55, further comprising calculating Ek[|mx(k)|] and Ek[|my(k)|] in a coadaptive process from the movements mx(k) and my(k) respectively calculated in a most recent complete block of object movements.

57. The method according to claim 55, further comprising calculating Ek[|mx(k)|] and Ek[|my(k)|], a noncoadaptive process, by separately averaging mxk) and my(k) values calculated over a recent interval and updating this value regularly.

58. The method according to claim 19, wherein, in step (b), at least three dimensions of the object's movements are calculated on one (x), another (y) and a further (z) axis at time t as:
ΣᵢWx(n_or_{—}p)i*NRᵢ(t)=X(t),
ΣᵢWy(n_or_{—}p)i*NRᵢ(t)=Y(t),
and
ΣᵢWz(n_or_{—}p)i*NRᵢ(t)=Z(t),
(i) where the index, i, refers to each of a plurality of electrical input signals derived from detected electrical impulses, and the values are summed over all signals beingused,
(ii) NRᵢ(t) is the normalized input signal,
(iii) Wxni is the x axis negative weighting factor used if NRᵢ(t)<0, and Wxpi is the x axis positive weighting factor used if NRᵢ(t)>0,
(iv) Wyni is the y axis negative weighting factor used if NRᵢ(t)<0, and Wypi is the y axis positive weighting factor used if NRᵢ(t)>0,
(v) Wzni is the z axis negative weighting factor used if NRᵢ(t)<0, and Wzpi is the z axis positive weighting factor used if NRᵢ(t)>0.

59. The method according to claim 58, further comprising correction of X(t), Y(t) and Z(t) for drift including calculating the predicted movement in x, y and z at time t:
mx(t)=X(t)−Drift(t),
my(t)=Y(t)−Drift(t),
and
mz(t)=Z(t)−Drift(t)
where
(i) rift(t) in the X axis is estimated as Σᵢ(Wxpi−Wxni)*Eₖ[|NRᵢ(k)|]/2,
(ii) Drift(t) in the Y axis is estimated as Σᵢ(Wypi−Wyni)*Eₖ[|NRᵢ(k)|]/2,
(iii) Drift(t) in the Z axis is estimated as Σᵢ(Wzpi−Wzni)*Eₖ[|NRᵢ(k)|]/2,
(iv) Eₖ[|NRᵢ(k)|] is the expected value of |Nkᵢ(k)|, the absolute value of input signal i's normalized value.

60. The method according to claim 59, wherein step (b) further comprises calculating Eₖ[|NRᵢ(k)|] from the normalized input signals observed in a most recent complete block of object movements based one or more detected electrical input signals indicated by the index, i.

61. The method according to claim 59, wherein step (b) further comprises calculating Eₖ[|NRᵢ(k)|], in a noncoadaptive process, by averaging |NRᵢ(k)| over a recent interval and updating this value regularly.

62. The method according to claim 59, further comprising normalizing movement magnitudes of movement in time t of movement dimension m(t), in each of three dimensions (x, y and z) by applying:
Mx(t)=Vscale*mx(t)/Eₖ[|mx(k)|],
My(t)=Vscale*my(t)/Eₖ[|my(k)|],
and
Mz(t)=Vscale*mz(t)/Eₖ[|mz(k)|]
where Ek[|mx(k)|] is the expected value of the absolute value of mx(k) taken over all calculation times, k, for the x dimension, Ek[|my(k)|] is the expected value of the absolute value of my(k) taken over all calculation times, k, for the y dimension, and Ek[|mz(k)|] is the expected value of the absolute value of mz(k) taken over all calculation times, k, for the z dimension.

63. The method according to 62, further comprising calculating Ek[|mx(k)|], Ek[|my(k)|] and Ek[|mz(k)|] in a coadaptive process from the movements mx(k), my(k), and mz(k) respectively calculated in a most recent complete block of object movements.

64. The method according to 62, further comprising calculating Ek[|mx(k)|], Ek[|my(k)|], and Ek[|mz(k)|], in a noncoadaptive process, by separately averaging mx(k), my(k) and mz(k) values calculated over a recent interval and updating this value regularly.

65. The method according to claim 33, further including the step of adaptation comprising presenting to the subject targets to which the object is to be moved, in blocks of target-pursuing tasks, calculating movements in at least one dimension, Mσj(t) for a completed block, and adjusting at least one of the weights Wσjpi, Wσjni, in a manner that would have improved target pursuit in at least one of the target-pursuing tasks.

66. The method according to claim 65, wherein adjusting at least one of the weights includes determining at least one of the weights pursuant to the following equation for at least one value of j indicating the dimensions 1 through N:
Wσⱼpi(S+1)=Bσⱼₚᵢ(P_{S}(Wσⱼpi(S)−AσⱼₚᵢΔWσⱼpi(S))+(1−P_{S})Wσⱼpi(S_{best})),
or
Wσⱼni(S+1)=Bσⱼₙᵢ(Pₛ(Wσⱼni(S)−AσⱼₙᵢΔWσⱼni(S))+(1−Pₛ)Wσⱼni(S_{best})),
the weights for the next block being partly based on the current weights, adjusted for errors seen in most recent block, S, and partly based on the weights that produced the best results over the last Q blocks of object movements, where Q is an integer of 2 or greater. Ps is a value between 0 and 1 and indicates the proportion of the weights in the next block, which should be based on the weights in the current block and the remaining proportion of the weights in the next block is based on the weights from the block out of the last Q blocks where the resulting movements were the most desirable.

67. A specific embodiment of claim 66, were Ps is:
Pₛ=(1−Phit(S_{best})/(Phit(S_{best})+Phit(S)+q))*(1−(Phit(S_{best})−Phit (S))),
where Phit( ) is a measure of the quality of movements in a given block and is between 0 and 1, S is the most recent block of movements and Sbest is the block out of the last Q blocks which had the highest quality of movements, and q is a very. small number used to prevent dividing by zero.

68. The method according to claim 66, were Ps is any monotonic function where Pₛ goes toward 0 as Phit(S_{best})>>Phit (S) and goes to ˜0.5 as Phit(S) goes toward Phit(S_{best}).

69. The method according to claim 66, where the best block, S_{best} is determined by first, the highest number of correct movements made in the block, and, if there's a tie between blocks, secondly, the block in which the movements were made the fastest.

70. The method according to 66, where at least one of the adjustments to the weights in one or more of the dimensions 1 through N, ΔWσⱼpi(S), ΔWσⱼni(S), are a function of the errors seen during the most recently completed block S.

71. The method according to 66, where at least at least one of the adjustments to the weights in one or more of the dimensions 1 through N, ΔWσⱼpi(S), ΔWσⱼni(S),is such that one or more of the adjusted weights, calculated as:
(Wσⱼpi(S)−A_{σjpi}ΔWσⱼpi(S)),
and
(Wσⱼni(S)−A_{σjni}ΔWσⱼni(S)),
would result in a new value of Wσⱼ(p_or_n)i which would have reduced the movement error in at least one dimension seen in movement block S.

72. The method according to 66 where at least one positive weight adjustment in at least one dimension, ΔWσⱼpi (S) is calculated as:
ΔWσⱼpi(S)=Eₖ[Wσⱼpi(k)NRᵢ(k)−(Tσⱼ(k)−Cσⱼ(k))],
where the expected value, E[], is taken over just the time steps, k, in block S during which the normalized rate, NRᵢ(k), is positive, Tσⱼ(k) is the desired movement in the σⱼ dimension and Cσⱼ(k) is the actual value in the σⱼ dimension of the brain-controlled object being moved at time k, at least one negative weight adjustment in at least one dimension ΔWσⱼni(S) is calculates as:
ΔWσⱼni(S)=Eₖ[Wσⱼni(k)NRᵢ(k)−Tσⱼ(k)−Cσⱼ(k))]
where the expected value, E[], is taken over just the time steps, k, in block S during which the normalized rate, NRᵢ(k), is negative Tσⱼ(k) is the target or desired movement in the σⱼ dimension and Cσⱼ(k) is the actual value in the σⱼ dimension of the brain-controlled object being moved at time k.

73. A method according to claim 66, where Aσⱼₚᵢ is a positive value chosen to control how much the weight, Wσⱼpi, is changed between each block of movements, and Aσⱼₙᵢ is also a positive value chosen to control how much the weight, Wσⱼni, is changed between each block of movements.

74. A method according to claim 73, where at least one Aσⱼpi for at least one dimension is calculated as:
Aσ_{j pi}=Aₒ(1+C_{A1}(N[EMσⱼpi(S)]+N[ECσⱼpi(S)])),
where
(i) Aₒ=Amax−(EQ[Phit(Q)]C_{A2} Aₘₐₓ), and E_{Q}[Phit(Q)]is a measure of the quality of movements over the last Q blocks of movements,
(ii) Aₘₐₓ. C_{A1} and C_{A2} are constants,
(iii) C_{A2} is between 1 and 0, and sets the minimum Aₒ to (1−C_{A2})Aₘₐₓ,
(iv) N[EMσⱼpi(S)] is a normalized value which is a function of the magnitude of the movement errors in dimensions σⱼ during movement block S when the normalized firing value of input signal i was positive,
(v) N[ECσⱼpi(S)] is a normalized value which is a function of the consistency of the movement errors in dimensions σⱼ during movement block S when the normalized value of input signal i was positive.

75. A method according to claim 73, where at least one Aσⱼni for at least one dimension is calculated as:
Aσ_{j ni}=Aₒ(1+C_{A1}(N[EMσⱼni(S)]+N[ECσⱼni(S)])),
where
(i) Aₒ=Aₘₐₓ−(E_{Q}[Phit(Q)]C_{A2} Aₘₐₓ), and E_{Q}[Phit(Q)]is a measure of the quality of movements over the last Q blocks of movements,
(ii) Aₘₐₓ, C_{A1} and C_{A2} are constants,
(iii) C_{A2} is between 1 and 0, and sets the minimum Aₒ to (1−C_{A2})Aₘₐₓ,
(iv) N[EMσⱼni(S)] is a normalized value which is a function of the magnitude of the movement errors in dimensions σⱼ during movement block S when the normalized value of input signal i was negative,
(v) N[ECσⱼni(S)] is a normalized value which is a function of the consistency of the movement errors in dimensions σⱼ during movement block S when the normalized value of input signal i was negative.

76. The method according to 74 wherein E_{Q}[Phit(Q)] is the average proportion of the movements which went to the correct targets during blocks Q.

77. The method according to claim 75 wherein E_{Q}[Phit(Q)] is the average proportion of the movements which went to the correct targets during blocks Q.

78. The method according to claim 74 wherein EMσⱼpi(S) and ECσⱼni(S) are calculated as:
EMσⱼpi(S)=|Eₖ[Wσⱼpi(k) NRi(k)−(Tσⱼ(k)−Cσⱼ(k))]|

ECσⱼpi(S)=EMσⱼpi(S)/Eₖ[|Wσⱼpi(k)NRi(k)−(Tσⱼ(k)−Cσⱼ(k))|],
where
(i) | . . . | represents the absolute value,
(ii) Eₖ[ . . . ] represent the expected value over all times in k where the normalized input signal, NRi(k), was above zero,
(iii) Tσⱼ(k) is the desired movement in the σⱼ dimension and Cσⱼ(k) is the actual value in the σⱼ dimension of the brain-controlled object being moved at time k.

79. The method according to claim 75 wherein EMσⱼni(S) and ECσⱼni(S) are calculated as:
EMσⱼni(S)=|Eₖ[Wσⱼni(k)NRi(k)−(Tσⱼ(k)−Cσⱼ(k))]|

ECσⱼni(S)=EMσⱼni(S)/Eₖ[|Wσⱼni(k) NRi(k)−(Tσⱼ(k)−Cσⱼ(k))|]
where
(i) | . . . | represents the absolute value,
(ii) Eₖ[ . . . ] represent the expected value over all times in k where the normalized input signal, NRi(k), was below zero,
(iii) Tσⱼ(k) is the desired movement in the σⱼ dimension and Cσⱼ(k) is the actual value in the σⱼ dimension of the brain-controlled object being moved at time k.

80. The method according to claim 74, wherein N[ . . . ] normalizes the enclosed terms across all input signals, i, to between −1 and 1.

81. The method according to claim 75, wherein N[ . . . ] normalizes the enclosed terms across all input signals, i, to between −1 and 1.

82. The method according to claim 74, wherein N[ . . . ] normalizes the enclosed terms across all input signals, i, to between −1 and 1 by:
(i) subtracting the mean on the enclosed terms taken across all i,
(ii) dividing by two standard deviations of the enclosed terms taken across all i,
(iii) truncating to −1 or 1 any values which are outside of the range −1 to 1.

83. The method according to claim 75, wherein N[ . . . ] normalizes the enclosed terms across all input signals, i, to between −1 and 1 by:
(i) subtracting the mean on the enclosed terms taken across all i,
(ii) dividing by two standard deviations of the enclosed terms taken across all i,
(iii) truncating to −1 or 1 any values which are outside of the range −1 to 1.

84. The method according to claim 66, wherein at least at least one of the adjustments to the positive or negative weights in one or more of the dimensions 1 through N, (Bσⱼₚᵢ, Bσⱼₚᵢ) is such that the input signal, i's, comparable weight in the next block (S+1) will be scaled up or down as a function of how useful that signal has been in controlling movement in one or more of those dimensions of movement.

85. The method according to claim 66, wherein at least one of the adjustments to the positive or negative weights in one or more of the dimensions 1 through N, (Bσⱼₚᵢ, Bσⱼₚᵢ) is determined by the functions:
Bσⱼₚᵢ=1+C_{B}Bₒ(N[ECσⱼpi(S)]−N[EMσⱼpi(S)])
or
Bσⱼₙᵢ=1+C_{B}Bₒ(N[ECσⱼni(S)]−N[EMσⱼni(S)])
where
(i) Bₒ=1−E_{Q}[Phit(Q)], and E_{Q}[Phit(Q)] is a function of the movement quality over the previous Q movement blocks with a value in the range from 0 to 1,
(ii) CB is a positive constant,
(iii) N[ECσⱼpi(S)] and N[ECσⱼni(S)] are normalized measures of the consistency of the movement errors in dimension σⱼ attributed to input signal, i, when signal i's normalized value is above or below zero respectively, and
(iv) N[EMσⱼpi(S)] and N[EMσⱼni(S)] are normalized measures of the magnitude of the movement errors in dimension σⱼ attributed to input signal, i, when signal i's normalized value is above or below zero respectively.

86. The method according to claim 74 or 75 wherein E_{Q}[Phit(Q)] is the average proportion of the movements that reached their intended targets during the most recent Q movement blocks.

87. A method according to claim 74, where at least one Aσⱼni for at least one dimension is calculated as:
Aσ_{j ni}=Aₒ(1+C_{A1}(N[EMσⱼni(S)]+N[ECσⱼni(S)])),
where
(i) Aₒ=Aₘₐₓ−(E_{Q}[Phit(Q)] C_{A2} Aₘₐₓ and E_{Q}[Phit(Q)]is a measure of the quality of movements over the last Q blocks of movements,
(ii) Aₘₐₓ, C_{A1} and C_{A2} are constants,
(iii) C_{A2} is between 1 and 0, and sets the minimum Aₒ to (1−C_{A2})Aₘₐₓ,
(iv) N[EMσⱼni(S)] is a normalized value which is a function of the magnitude of the movement errors in dimensions σⱼ during movement block S when the normalized value of input signal i was negative,
(v) N[ECσⱼni(S)] is a normalized value which is a function of the consistency of the movement errors in dimensions σⱼ during movement block S when the normalized value of input signal i was negative.

88. The method according to claim 66, wherein EMσⱼpi(S) and ECσⱼpi(S) are calculated as:
EMσⱼpi(S)=|Eₖ[Wσⱼpi(k)NRi(k)−(Tσⱼ(k)−Cσⱼ(k))]51
ECσⱼpi(S)=EMσⱼpi(S)/Eₖ[|Wσⱼpi(k)NRi(k)−(Tσⱼ(k)−Cσⱼ(k))|]
and
EMσⱼni(S) and ECσⱼni(S) are calculated as:
EMσⱼni(S)=|Eₖ[Wσⱼni(k)NRi(k)−(Tσⱼ(k)−Cσⱼ(k))]|

ECσⱼni(S)=EMσⱼni(S)/Eₖ[|Wσⱼni(k) NRi(k)−(Tσⱼ(k)−Cσⱼ(k))|]
where
(i) | . . . | represents the absolute value,
(ii) Eₖ[ . . . ] represent the expected value over all times in k where the normalized input signal, NRi(k), was above zero when used with equations containing Wσⱼpi(k), and NRi(k), was above zero when used with equations containing Wσⱼni(k),
(iii) Tσⱼ(k) is the desired movement in the σⱼ dimension and Cσⱼ(k) is the actual value in the σⱼ dimension of the brain-controlled object being moved at time k.

89. The method according to claim 66, wherein N[ . . . ] normalizes the enclosed terms across all input signals, i, to between −1 and 1.

90. The method according to claim 66, wherein N[ . . . ] normalizes the enclosed terms across all input signals, i, to between −1 and 1 by:
(i) subtracting the mean of the enclosed terms taken across all i,
(ii) dividing by two standard deviations of the enclosed terms taken across all i,
(iii) truncating to −1 or 1 any values which are outside of the range −1 to 1.

91. An electrical controller comprising a computational processor programmed to operate as defined in any one of claims 19-90.

92. Programming for a computational processor having routines for effecting the method of any one of claims 19-90.

93. A brain neuron activated control system, including:
(a) an array of thin, closely spaced conductive electrodes adapted to enter an animal's brain, each operative to receive electrical impulses from a brain location comprising one or more neurons,
(b) a programmable computer,
(c) a plurality of electrical conductors, each conductor connected to one or more of the electrodes for conducting the electrical impulses received by an electrode to an input interface to the computer,
(d) a visible computer output display device coupled to the computer,
(e) programming for operating the computer, including:
i) programming operative to create at least one moveable object in the display;
ii) object control programming responsive to the electrical signals received at the interface to cause the movement of the moveable object in the display, said object control programming comprising:
a program to calculate movement of the moveable object in at least one dimension, first by calculating a normalized firing rate, NRᵢ(t), in a time window, by each of the locations producing impulses in the electrodes and, second, weighting a firing rate-related value for at least a portion of the firing rates by a positive weighting factor if NRᵢ(t) was greater than a mean firing rate and by a negative weighting factor if the Nrᵢ(t) was less than the mean firing rate, and moving the moveable object in the display a distance dependent upon at least a portion of the weighted firing rate-related values.
